(19) United States
(12) Patent Application Publication
(10) Pub. No.: US 2016/0089456 A1
(43) Pub. Date: Mar. 31, 2016

(54) METHODS DEVICES AND SYSTEMS OF PREPARING TARGETED MICROBUBBLE SHELLS

(71) Applicant: The Trustees of Columbia University in the City of New York, New York, NY (US)

(72) Inventors: Mark A. BORDEN, Boulder, CO (US); Cherry CHEN, New York, NY (US)

(21) Appl. No.: 14/868,709

(22) Filed: Sep. 29, 2015

Related U.S. Application Data
(62) Division of application No. 13/695,677, filed on Dec. 27, 2012, now abandoned.
(60) Provisional application No. 61/330,331, filed on May 1, 2010.

(51) Int. Cl. A61K 49/22

(52) U.S. Cl. CPC A61K 49/223; A61K 49/221

(57) ABSTRACT

Targeted microbubbles are generated by post-labeling buried ligand microbubbles. According to embodiments, buried ligand microbubbles are created with steric brushes protecting functionalized polymer tethers. Ligands were attached to the functionalized tethers by diffusion of ligands of a small size through the steric barrier. The steric barrier was substantially capable of hindering access to the tethers by larger molecules. The embodiments disclosed include methods for creating microbubble batches that can be loaded with selected ligands, for titrating the targeting ligands thereby to reduce waste and cost, and for using resulting buried ligand molecules for medical purposes.

This invention was made with government support under R01 EB 009066 awarded by National Institute of Health. The government has certain rights in the invention.

### BACKGROUND

A microbubble may be, for example, a gaseous colloidal particle with diameter less than 10 μm, of which the surface comprises amphiphilic phospholipids self-assembled to form a lipid monolayer shell. Due to the compressible gas core, microbubbles may provide a sensitive acoustic response and are currently used as ultrasound contrast agents. Similar to the design of long circulating liposomes, poly(ethylene glycol) (PEG) chains are typically incorporated into the shell of microbubbles to form a steric barrier against coalescence and adsorption of other macromolecules to the microbubble surface.

Fabricated three-dimensional (3D) extracellular matrices (ECMs) can be used to mimic the often in homogeneous and anisotropic properties of native tissues and to construct in vitro cellular environments. Since these 3D ECMs provide physiologically relevant cellular environments, they can be used to study tissue morphogenesis as well as to engineer tissue. For example, 3D collagen and fibrin matrices can be used for analyzing the mechanisms of epithelial branching morphogenesis and endothelial cell capillary morphogenesis as well as for engineering vascular and cardiac tissues. To this end, bulk isotropic 3D matrices have been employed in which cells are randomly dispersed. However, these bulk structures offer limited capacity for cell stimulation, providing nutrients, control of growth factors and other features of living tissue.

A microbubble is a gaseous colloidal particle with diameter less than 10 μm, of which the surface comprises amphiphilic phospholipids self-assembled to form a lipid monolayer shell. Due to the compressible gas core, microbubbles may provide a sensitive acoustic response and are currently used as ultrasound contrast agents.

### SUMMARY

Complement fixation to surface-conjugated ligands plays a critical role in determining the fate of targeted colloidal particles after intravenous injection. The immunogenicity of targeted microbubbles with various surface architectures and ligand surface densities was demonstrated using a novel flow cytometry technique. Methods devices and systems for targeted microbubbles generation employ a post-labeling technique with a physiological targeting ligand. An embodiment employs as a ligand, cyclic arginine-glycine-asparagine (RGD) which, according to embodiments, is attached to the distal end of the poly(ethylene glycol) (PEG) moieties on the microbubble surface Microbubbles. To demonstrate immune response, microbubbles were incubated in human serum, washed and then mixed with fluorescent antibodies specific for various serum components. It was found that complement C3/C3b was the main human serum factor to bind in vitro to the microbubble surface, compared to IgG or albumin. The PEG brush architecture on C3/C3b fixation to the microbubble surface RGD peptide was able to trigger a complement immune response, and complement C3/C3b fixation depended on microbubble size and RGD peptide surface density. When the targeting ligand was attached to shorter PEG chains that were shielded by a PEG overbrush layer (buried-ligand architecture), significantly less complement activation was observed when compared to the more traditional exposed-ligand motif. The extent of this protective role by the PEG chains depended on the overbrush length. Taken together, the results confirm that the buried-ligand architecture may significantly reduce ligand-mediated immunogenicity.

Methods, devices, and systems are described for forming, storing, and utilizing microbubbles with active surfaces. For example, microbubbles are characterized by a buried ligand architecture, which is described. In embodiments, microbubbles are prepared and formed into a storable material and to make them ready to be converted into buried ligand structures by post labeling. Various other embodiments are described.

### BRIEF DESCRIPTION OF FIGURES

FIG. 1 (A) is an illustration of the molecular structure of NHS-FITC showing its estimated dimensions using the Stokes-Einstein equation for the diffusion of a sphere in a liquid. The diffusion constant of a free FITC molecule at 21.5 C in water was calculated to be 0.49×10-9 m2/s, and the dynamic viscosity of water was estimated to be 0.979×10−3 kg/m·s.

FIG. 1B is a schematic diagram of streptavidin-FITC showing its estimated dimensions.

FIG. 1C is a figurative representation of the dimensions of a microbubble with either exposed- or buried-ligand architecture (ELA or BLA). The PEG chain length was estimated using self-consistent field (SCF) theory using values of 0.44 nm2 for the average projected area per lipid molecule and 0.35 nm for PEG monomer length. The lipid monolayer thickness was estimated to be ˜3 nm based on the persistence length of the stearoyl chains.

FIG. 2A shows microbubble size isolation and flow cytometry gate determination for number-weighted and volume-weighted.

FIG. 2B shows microbubble size distributions before and after size isolation where each curve is the average of three measurements with its SD plotted as error bars.

FIG. 2C shows FSC vs. SSC plots of corresponding microbubble samples before and after size isolation where a tight fitted P gate and a rectangular R gate was drawn for each scatter plot and saved as templates for all subsequent measurement in order to identify each size subpopulations in a polydisperse suspension.

FIG. 3A shows size distribution of the DiI-labeled microbubble suspension after partially removing 1-2 μm population. The 1-2 μm and 4-5 μm peaks shown in the number % size distribution were of similar magnitude to ensure proper event detection using flow cytometry.

FIG. 3B is an FSC vs. SSC density plot of the same microbubble suspension.

FIG. 3C is an MFI histogram of the microbubble suspension of FIG. 3B, showing a multimodal distribution that corresponded to the Accusizer measurement.

FIG. 4A shows a typical flow cytometry fluorescence intensity histogram of microbubbles with different architectures before and after ligand conjugation for NHS-FITC ELA binding.

FIG. 4B shows a typical flow cytometry fluorescence intensity histogram of microbubbles with different architectures before and after ligand conjugation for SA-FITC ELA binding.

FIG. 4C shows a typical flow cytometry fluorescence intensity histogram of microbubbles with different architectures before and after ligand conjugation for NHS-FITC BLA binding.

FIG. 4D shows a typical flow cytometry fluorescence intensity histogram of microbubbles with different architectures before and after ligand conjugation for SA-FITC BLA binding.

FIG. 5A shows optimization of ligand:functionalized lipid ratio where MFI was measured before and after microbubble samples reacted with various amounts of ligands after 12 hours at room temperature for NHS-FITC:DSPE-PEG2000-A with molar ratio varied between 0.04 and 100 wherein 20 molar ratio (dash line) was used for all subsequent kinetics studies.

FIG. 5B shows optimization of ligand:functionalized lipid ratio where MFI was measured before and after microbubble samples reacted with various amounts of ligands after 12 hours at room temperature for SA-FITC:DSPE-PEG2000-B with molar ratio varied between 0.01 and 1.5. 0.5 molar ratio (dash line) was used for all subsequent kinetics studies.

FIG. 6. shows NHS-FITC binding kinetics to the tethered amino functional groups after microbubble formation where MFI was monitored continuously over 6 hours, and MFI change before and after reaction for each size range was plotted at different time points. Data was fitted using a pseudo-first order reaction kinetics model showing for ELA and BLA good agreement for both the observed binding rate and the final MFI over the experimental time scale.

FIG. 7 shows SA-FITC binding kinetics to the tethered biotin functional groups after microbubble formation in which ELA reached saturation binding within the first 10 min of reaction with BLA showing gradual increase of MFI over the first 2 hours of reaction with a half-time around 30 min for all size ranges; the difference in binding rate indicating that the PEG overbrush interferes with the diffusion of large molecules to the surface of microbubbles and partially blocks their binding to the buried end groups.

FIGS. 8A and 8C compares normalized MFI change between ELA and BLA for all size ranges where the last measured MFI was used for normalizations as the saturation value and the curves were obtained using a pseudo-first order kinetics model for normalized MFI change of NHS-FITC binding for ELA and BLA microbubbles where the fitted binding rate for all size ranges was the same for each condition, indicating that the ligand binding rate was independent of microbubble size.

FIGS. 8B and 8D compares normalized MFI change between ELA and BLA for all size ranges where the last measured MFI was used for normalizations as the saturation value and the curves were obtained using a pseudo-first order kinetics model for normalized MFI change of SA-FITC binding for ELA and BLA microbubbles where the fitted binding rate for all size ranges was the same for each condition, indicating that the ligand binding rate was independent of microbubble size.

FIG. 9A shows a sample comparison between normalized MFI change for ELA and BLA 1-2 μm microbubbles with the binding rate for NHS-FITC; ELA and BLA being the same and showing that the diffusion and attachment of small molecules to the tethered short PEG chains was not affected by the overbrush and showing that the binding rate for SA-FITC between ELA and BLA was significantly different, particularly during the first 30 min of reaction, indicating that the binding of large SA-FITC molecules was slowed by the PEG overbrush in BLA.

FIG. 9B shows a sample comparison between normalized MFI change for ELA and BLA 1-2 μm microbubbles with the binding rate for SA-FITC; ELA and BLA being the same and showing that the diffusion and attachment of small molecules to the tethered short PEG chains was not affected by the overbrush and showing that the binding rate for SA-FITC between ELA and BLA was significantly different, particularly during the first 30 min of reaction, indicating that the binding of large SA-FITC molecules was slowed by the PEG overbrush in BLA.

FIG. 10A is an Epi-fluorescence image of microbubble samples after ligand binding in which arrows point to microstructural features of non-uniform NHS-FITC labeling of ELA microbubbles and scale bars indicate 10 μm.

FIG. 10B is an Epi-fluorescence image of microbubble samples after ligand binding in which arrows point to microstructural features of non-uniform NHS-FITC labeling of BLA microbubbles and scale bars indicate 10 μm.

FIG. 10C is an Epi-fluorescence image of microbubble samples after ligand binding in which arrows point to microstructural features of non-uniform SA-FITC labeling of ELA microbubbles and scale bars indicate 10 μm.

FIG. 10D is an Epi-fluorescence image of microbubble samples after ligand binding in which arrows point to microstructural features of non-uniform SA-FITC labeling of BLA microbubbles and scale bars indicate 10 μm.

FIG. 11 illustrates possible phase separation between lipopolymer species on the surface of microbubbles.

FIG. 12. shows, for flow cytometric identification of surface structure induced by streptavidin binding, the change in the percentage of events that fell within the serpentine P gate and the accompanying FSC vs. SSC plots.

FIG. 13A are Epi-fluorescence images of typical microbubbles showing attached surface structures (domains, folds, protrusions) in which the scale bar corresponds to 10 μm.

FIG. 13B is a figurative representation illustrating possible streptavidin-induced monolayer protrusion.

FIG. 14 shows concentration change for ELA and BLA microbubbles upon SA-FITC binding during 6 h. where concentration date were obtained from flow cytometry data using the tight-fitted P gates.

FIGS. 15A and 15B represent 5% RGD peptide labeled microbubble size distribution change during human complement-preserved serum incubation at 37° C. where the size distribution was continuously monitored for 2 hours for both ELA (15A) and BLA (15B) microbubble samples; with the exception that some smaller microbubbles (diameter <2 μm) showed a decrease in number detected over time, the majority of targeted microbubbles were stable during incubation with no significant change in size.

FIGS. 15C and 15D represent the total concentration as measured by Accusizer (15C) and flow cytometer (15D) and is plotted against time according to microbubble diameter ranges; both techniques showing data in agreement; even though a concentration decrease was observed for both designs at the end of 2-hour incubation time, more than 30% of the targeted microbubbles were stable at 30 min, which was in the same time scale as for a typical ultrasound contrast imaging session.

FIG. 16 ELISA shows results of complement component C3/C3b activity for human complement-preserved serum aliquots Serum aliquots were randomly chosen to be tested throughout the immunogenicity experiments The average of measured C3/C3b activity was 30±16 μg/mL of serum (mean±SD) The human serum samples from different batches were statistically identical in terms of complement C3/C3b activity

FIG. 17 shows human serum factor binding to 5% RGD labeled 1-2 μm ELA and BLA microbubbles where the median fluorescence intensity (MFI) was measured after RGD peptide conjugation, after 2 hours human serum incubation and after 1 hour anti-human serum factor FITC-antibodies incubation and where all three serum factors were observed to bind to both targeted microbubbles. Only complement C3/C3b showed significant MFI increases; while IgG and albumin showed much less binding “*” denotes a significant increase vs the corresponding “RGD+Serum” measurement (p<005)

FIGS. 18A and 18B show microscopic images of 5% RGD labeled ELA microbubbles after C3/C3b binding in both bright field mode (18A) and epifluorescence mode (a8B). Both images show the same field of view. The enlarged images (for example as shown at 1802) indicate microstructural features of non-uniform C3/C3b binding. Scale bars correspond to 10 μm.

FIG. 19 represents human complement C3/C3b binding to control microbubbles P2K/P5K control microbubbles showed significant lower C3/C3b binding than P2K control in all microbubble size ranges P5K control microbubbles showed the lowest amount of C3/C3b binding, suggesting a thicker and denser protective layer was formed by the DSPE-PEG5000 chains than either DSPE-PEG2000 or DSPE-PEG2000/5000 mixture “*” denotes a significant difference vs the corresponding P2K control, and “#” denotes a significant difference vs the corresponding P5K control (p<005).

FIGS. 20A and 20B represent RGD surface coverage and size dependence of complement C3/C3b binding to targeted ELA (FIG. 20A) and BLA (FIG. 20B) microbubbles, For ELA microbubbles, higher RGD surface coverage led to more complement C3/C3b binding But for BLA microbubbles, the PEG overbrush successfully protected the RGD peptide; no significant increase of MFI values was detected when the RGD conjugation amount was increased by two orders of magnitude. For both surface architectures, large targeted microbubbles (6-8 μm) showed significantly higher C3/C3b binding than smaller ones (1-2 μm and 4-5 μm), possibly due to the higher amount of RGD peptide presented on the surface “*” denotes a significant increase vs the corresponding 4-5 μm sample with 5% RGD, and “#” denotes a significant increase vs the corresponding 6-8 μm sample with 05% RGD (p<005).

FIG. 21 represents flow cytometric analysis of complement C3/C3b binding to 5% RGD labeled 1-2 μm ELA and BLA microbubbles. Significant binding occurred to RGD labeled ELA microbubbles in comparison with P2K control, suggesting the targeting ligand was immunogenic BLA microbubbles also showed C3/C3b binding, indicating partial complement protein fixation. However, the increase of MFI value for targeted ELA microbubbles was much higher than that for BLA microbubbles, indicating that the buried-ligand architecture may shield the RGD peptide from complement recognition “*” denotes a significant increase vs the corresponding “RGD+Serum” measurement, and “#” denotes a significant increase vs BLA 5% (p<005).

FIG. 22 shows the effect of PEG overbrush length on complement C3/C3b fixation. For targeted microbubbles, BLA-P3K showed an intermediate MFI value, supporting the hypothesis that DSPE-PEG3000 chains formed an intermediate brush layer to protect the targeting ligand from the complement system “*” denotes a significant difference vs the corresponding ELA 5% (p<005).

FIG. 23 shows the effect of surface charge on complement C3/C3b fixation. Higher negative zeta potential led to a higher complement C3/C3b binding, suggesting a weak correlation between microbubble surface charge and complement activation

### DETAILED DESCRIPTION

When administered intravenously, microbubbles or other conventional colloidal particles are rapidly removed from the bloodstream by animal (e.g., human) immune system. This may be triggered by receptor recognition, and such ligand-receptor interactions. Serum protein adsorption plays a role in determining particle uptake by phagocytes and predicting the fate of colloidal particles after administration Immunoglobulin G (IgG) and complement components are known opsonins for the uptake of large particles, such as bacteria, viruses, and remnants of dead cells. Complement activation plays a critical role in the recognition of biocolloids by the immune system.

The complement system, consisting of over 30 soluble plasma and cell-surface bound proteins, is an important effector arm of innate immunity. There are multiple pathways to activate the complement system: the classical pathway, the lectin pathway and the alternative pathway. The classical pathway is triggered by the binding of complement component C1q to immune-complexes on the antigen surfaces; the lectin pathway is triggered by the binding of mannose-binding lectin to arrays of carbohydrates on foreign microorganisms; and the alternative pathway is triggered by the binding of spontaneously activated complement component C3 in plasma to the surface of foreign particles. All three pathways converge to the formation of C3 convertases, which cleave C3 into C3b and C3a for further opsonization and mediation of inflammation in the complement cascade. One key site for the activation of the complement system is the foreign particle surface. Regardless of the activation pathway, the main effectors of the complement system (such as C3 convertases and C3b) need to bind to the surface of the particle in order to initiate the phagocytic process. According to embodiments of the disclosed subject matter, the accessibility of the complement component proteins to the foreign particle surface is controlled to selectively inhibit complement activation as described herein.

Targeted microbubbles are created by attaching a targeting ligand, such as a polysaccharide, monoclonal antibody or peptide, specific for the desired endothelial biomarker, onto the shell. Cyclic-arginine-glycine-asparagine (RGD) has been shown to bind to an overexpressed angiogenic biomarker, αvβ3 integrin, with high affinity and specificity. Specific ligands may be attached to the distal end of tethered PEG chains. However, targeting ligands typically present nucleophilic groups (e.g., hydroxyl and amino) that could trigger the alternative pathway of complement activation and decrease the microbubble circulation persistence. Long circulating PEGylated liposomes, with similar surface structures to microbubbles, could trigger acute hypersensitivity reaction in sensitive individuals. These reactions are classified as complement activation-related pseudoallergy (CARPA) due to their common trigger mechanism: complement activation. PEGylated liposomes are capable of triggering the complement system in human serum and fixing opsonic complement proteins. Targeted microbubbles, according to embodiments, may be made with a surface architecture that minimizes complement recognition by minimizing C3/C3b fixation in order to reduce CARPA and prevent premature microbubble clearance from the circulatory system. At the same time, avoidance of complement fixation may keep the ligand pristine and therefore allow it to retain specificity to the target receptor.

According to embodiments, a microbubble construct for use with ultrasound radiation force (USRF) to allow triggered and specific adhesion with reduced immunogenicity is employed. The microbubble design, buried-ligand architecture (BLA), employs bimodal PEG polymer chains (two PEG chain lengths) on the surface of microbubbles. A targeting ligand is attached to the shorter PEG chains, while the longer PEG overbrush serves as a shield to inhibit ligand exposure and reduce the accessibility to opsonins. The BLA motif reduces the complement activated immune response in addition to prolonged circulation persistence. The targeted microbubble immunogenicity was demonstrated in vitro between various microbubble surface architectures. Exposed-ligand architecture (ELA) and BLA microbubbles were generated with different PEG brush configurations and amounts of targeting RGD peptide conjugated to the microbubble shell. Instead of using a pre-synthesized lipoprotein-peptide conjugate as one of the shell components, a post-labeling technique to conjugate RGD peptides to the tethered PEG chains after microbubble generation and isolation was used. By quantifying the amount of complement C3/C3b binding to the microbubbles after human serum incubation, it was shown that the buried-ligand design decreased microbubble immunogenicity in vitro.

According to embodiments, a buried-ligand architecture (BLA) design for microbubbles is characterized by a microbubble surface coated with a bimodal PEG brush. In a method, microbubbles may be generated and fluorescent ligands with different molecular weight conjugated to the tethered functional groups on the shorter PEG, while the longer PEG serve as a shield to protect these ligands from being exposed to the surrounding environment.

It was shown that BLA microbubbles partially prevented the binding of macromolecules (>10 kDa) to the tethers due to the steric hindrances of the PEG overbrush, while allowing the uninhibited attachment of small molecules (<1 kDa). Approximately less than 40% less fluorescein conjugated streptavidin (SA-FITC) bound BLA microbubbles compared to exposed-ligand architecture (ELA) microbubbles.

A phase separation between the lipid species on the surface leading to populations of revealed and concealed ligands. Ligand conjugation kinetics was independent of microbubble size regardless of ligand size or microbubble architecture. Streptavidin-induced surface structure formation was observed for ELA microbubbles, and it is proposed that this phenomenon may be correlated to flow cytometry scattering measurements. Using microbubbles as model systems, molecular diffusion and binding to colloidal surfaces in a bimodal PEG brush layer were examined. In an application embodiment, post-labeling for small-molecule ligand to BLA microbubbles may generate targeted ultrasound contrast agents.

Principles of molecular diffusion in a polymer brush characterizing reactions in a polymer-grafted flat surface also apply to curved surfaces such as those of a gas-lipid interface posed by the surface of a microbubble. In an embodiment, a microbubble is a gas-filled colloidal particle with diameter less than 10 μm, of which the surface comprises amphiphilic phospholipids self-assembled to form a lipid monolayer shell. Similar to the design of long-circulation liposomes, poly(ethylene glycol) (PEG) chains, or PEG chain derivatives, are typically incorporated into the shell of microbubbles in order to form a steric barrier against coalescence and adsorption of other macromolecules to the microbubble surface. The protective role of PEG is understood to result from a steric hindrance effect due to the polymer brush—each PEG chain forms an impermeable “cloud” over the microbubble surface, which prevents other molecules from diffusing into the brush layer. Small PEG mushrooms may retard the binding of fluorescently labeled avidin to biotinylated liposomes.

As the PEG concentration increases, the overall vesicle fluorescence intensity decreases, indicating that the binding of avidin is retarded or even completely prevented due to the presence of PEG. It has been shown using a surface force apparatus that the dominant force that stabilize liposomes with short polymer chains grafted on the surface is steric repulsion. Repulsive thermal fluctuation forces swamp short-ranged van der Waals attraction and longer-range electrostatic interactions, and provide a physical barrier around the bilayer to prevent close approach of other surfaces, improving in vivo circulation persistence through reduction in opsonization and vesicle aggregation. For small molecules the effect may not hold because small molecules can find a path through the excluded volume.

For drug targeting applications, it may be desirable to engineer a drug delivery vehicle that has both high specificity and low immunogenicity. There are many surface functionalization strategies for conjugating targeting ligands to colloidal particles (liposomes, microbubbles, nanoparticles etc.), most notably through attaching specific ligands to the distal end of tethered PEG chains. However, targeting ligands typically present chemical groups that could trigger immune activation and decrease the circulation persistence of these colloidal particles. A useful structure may employ a bimodal mixture of grafted PEG chains, that is, a fraction of shorter PEG bearing targeting ligands and a fraction of longer PEG without ligands to minimize undesired immune complement activation and nonspecific adhesion.

Studies have shown that vertical segregation between the segments of the shorter and longer polymer chains occurred irrespective of the molecular weight differences or composition in a bimodal polymer brush layer. When two bimodal polymer surfaces were compressed, this stratification persisted. Segregation of the free ends of longer and shorter chains has been demonstrated. It has also been shown that the structural properties of the shorter chains depended very little on the lengths of the longer chains when both are highly stretched.

Force microscopy experiments have shown=that adhesion to avidin coated glass beads fail when biotin ligands were tethered to short PEG chains buried in a longer PEG overbrush. It has also been shown that specific adhesion of microbubbles with a bimodal polymer brush may be partially prevented in hydrodynamic conditions in comparison to exposed-ligand architecture. Ligand accessibility may be reduced when targeting ligands are attached to the shorter PEG in a bimodal mixture of PEG chains, therefore reducing undesired immune response. The instant specification describes using microbubbles as a model system characterize molecular diffusion and binding to colloidal surfaces in a bimodal PEG brush layer.

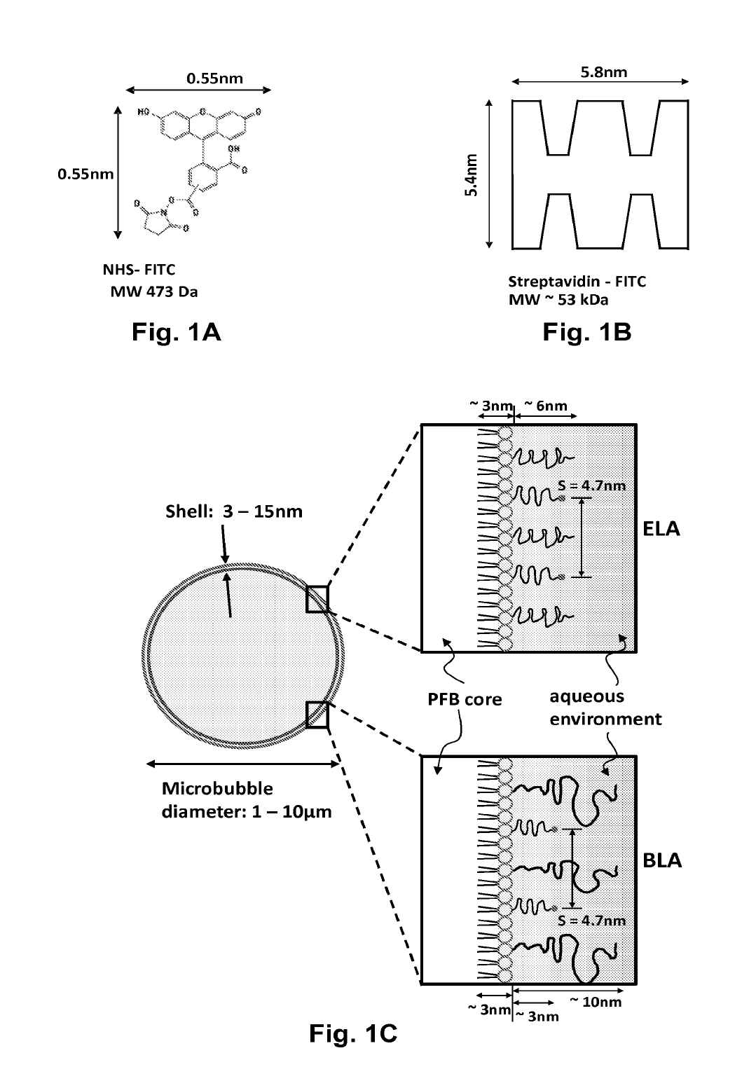

Due to the compressible gas core, microbubbles provide a sensitive acoustic response and are currently used as ultrasound contrast agents. When combined with targeting ligands, such as peptides, ultrasound allows the ultrasonic detection and evaluation of molecular biomarkers associated with intravascular pathology, including tumor angiogenesis, thrombosis and inflammation. To reduce the undesired immune response, a microbubble construct for use with ultrasound radiation force (USRF) may allow triggered and specific adhesion. This microbubble design may employ bimodal PEG polymer chains on the surface—the targeting ligand being attached to the shorter PEG chains (˜2000 Da) and hidden, with the longer PEG (˜5000 Da) overbrush serving as a shield to prevent ligand exposure. The structure reduces the complement activated immune response in addition to prolonged in vivo circulation persistence (FIG. 1C).

Buried-ligand microbubbles may be converted from stealth to active under USRF. That is, the shielded ligand may be revealed for binding only during microbubble oscillation in the acoustic field, but remain buried before and at the end of a USRF pulse. This buried-ligand architecture (BLA) design allowed spatial and temporal control of targeted adhesion. BLA microbubbles, compared to exposed-ligand architecture (ELA) microbubbles, may reduce immunogenicity without reducing targeted adhesion.

Microbubbles may be conjugated to a targeting ligand by either pre-labeling or post-labeling. Post-labeling benefits from the incorporation of functionalized lipids into the microbubble shell, and the targeting ligands are conjugated to the monolayer surface through either covalent bonds or noncovalent interactions after the microbubbles have been formed. This technique increases the efficiency of attaching targeting ligands since not all lipid molecules in a precursor liposomal mixture are ultimately incorporated into microbubble shells. This is particularly true for size-selected microbubbles. Instead of having a ligand attached to all lipid molecules, the amount of ligand needed for conjugation can be calculated from the microbubble concentration, size distribution and the area fraction of functionalized lipids of the microbubble suspension, thereby to optimize the cost of synthesis.

Post-labeling may also increase versatility by allowing multiple ligands to be conjugated to the same microbubble batch. This platform strategy for targeted contrast agent production increases safety, economy, and ease-of-use, and has other advantages over other techniques.

In order to utilize post-labeling for BLA microbubbles, the targeted ligand should be able to diffuse through the PEG overbrush and bind to the tethered functional groups at the surface. The PEG may partially prevent the diffusion and attachment of macromolecules. Polymer chains in solution may be highly dynamic due to thermal fluctuations. Their thermally driven conformational sampling property, or the breathing mode of the polymer chains, may strongly affect the ligand accessibility. Tethered molecules may extend well beyond their average equilibrium configuration over an experimental time scale of seconds, which broadens the overall spatial range of tethered ligand-receptor binding. For large molecules that are excluded from the brush layer due to steric hindrance, binding to the surface may still be possible due to transient excursions of polymer chains.

The differences in ligand diffusion and binding rate between various PEG brush architectures, ligand sizes and binding modes were experimentally tested. ELA and BLA microbubbles were generated to represent different polymer architectures. Solute size was varied by using 5/6-carboxyfluorescein succinimidyl ester (NHS-FITC) and fluorescein conjugated streptavidin (SA-FITC). NHS-FITC represents a class of smaller molecular ligands (<1 kDa), while SA-FITC represents a class of macromolelcular ligands (>10 kDa). By monitoring the fluorescence intensity change during binding, it was shown that BLA microbubbles partially prevented the binding of large molecules to the surface while allowing the uninhibited attachment of smaller ones. The findings provide information on binding of solutes to tethered groups in various brush architectures on a Langmuir monolayer-coated colloidal particle.

All phospholipids were purchased from Avanti Polar Lipids, Inc. (Alabaster, Ala.), including 1,2-distearyol-sn-glycero-3-phosphocholine (DSPC), 1,2-distearyol-sn-glycero-3-phosphoethanolamine-N-[methoxy(polyethylene glycol)2000] (DSPE-PEG2000), 1,2-distearyol-sn-glycero-3-phosphoethanolamine-N-[amino(polyethylene glycol)2000] (DSPE-PEG2000-A), 1,2-distearyol-sn-glycero-3-phosphoethanolamine-N-[biotinyl(polyethylene glycol)2000] (DSPE-PEG2000-B) and 1,2-distearyol-sn-glycero-3-phosphoethanolamine-N-[methoxy(polyethylene glycol)5000] (DSPE-PEG5000). The emulsifier polyoxyethylene-40 stearate (PEG40S) was purchased from Sigma-Aldrich (St. Louis, Mo.). All microbubble shell components were dissolved in chloroform (Sigma-Aldrich) and stored in the freezer at −20 C. The perfluorobutane gas (PFB, 99 wt % purity) used for microbubble generation was purchased from FluoroMed, L.P. (Round Rock, Tex.). The fluorophore probe 3,3′-dioctadecyloxacarbocyanine perchlorate (DiO) solution (Invitrogen; Eugene, Oreg.) was used to label microbubbles during the size-isolation experiment. NHS-FITC and SA-FITC were obtained from Pierce (Rockford, Ill.) and dissolved in N,N-dimethylformamide (DMF; Sigma-Aldrich) and 18 MΩ filtered deionized water (Direct-Q Millipore; Billerica, Mass.), respectively. Both solutions were stored at 4 C and discarded after 2 weeks.

Microbubbles used for size isolation and flow cytometry gate determination were made using DSPC and PEG40S at molar ratio 9:1. All other microbubble compositions used 90% DSPC and 10% DSPE-PEG (Table 1). The indicated amount of each lipid species was mixed in a separate vial, and chloroform was evaporated by flowing a steady stream of nitrogen over the vial during vortexing for about 10 minutes followed by several hours under house vacuum. 0.01 M phosphate buffer saline (PBS) solution (Sigma-Aldrich) was filtered using 0.2 μm pore size polycarbonate filters (VWR; West Chester, Pa.), and mixed with 10 vol % glycerol solution (Sigma-Aldrich) and 10 vol % 2-propanol solution (Sigma-Aldrich) to increase viscosity and lipid solubility. The dried lipid film was then hydrated with PBS mixture to a final lipid/surfactant concentration of 1 mg/mL.

Two methods of microbubble generation were used. For size isolation experiments, probe sonication was used. Briefly, the hydrated lipid mixture was first sonicated with a 20 kHz probe (Model 250A, Branson Ultrasonics, Danbury, Conn.) at low power (power setting dialed to 3/10; 3 W) to heat the lipid suspension above the DSPC main phase transition temperature (˜55° C.) and further disperse the lipid aggregates into small, unilamellar liposomes.31 1 mM DiO solution was added to the lipid suspension at an amount of 1 μL DiO solution per mL of lipid mixture. PFB was introduced by flowing it over the surface of the lipid suspension. Subsequently, high power sonication (power setting dialed to 10/10; 33 W) was applied to the suspension for about 10 s at the gas-liquid interface to generate microbubbles. No extra washing steps were done for size-isolation.

For the FITC-ligand binding tests, the shaking method was used to generate microbubbles. The lipid suspension was first heated to 60° C. in a digital heatblock (VWR) for 10 min, and then sonicated at 60° C. in a bath sonicator (Model 1510, Branson Ultrasonics; Danbury, Conn.) for 30 s so that the lipid aggregates were completely dispersed. 1 mM DiI solution was added to the lipid suspension at an amount of 1 μL DiI solution per mL of lipid mixture to generate fluorescently labeled microbubbles for the size analysis. 2 mL of lipid suspension was then transferred to a 3 mL serum vial and sealed for gas exchange. Gas exchange of the vial headspace was done by first vacuuming out air and then flowing PFB into the vial. At least three cycles of gas exchange were done to ensure the lipid suspension was saturated with PFB, and subsequently a 27 G needle was used to vent the vial in order to release the excess pressure. Microbubbles were formed by shaking with a VialMix (ImaRx Therapeutics; Tucson, Ariz.) for 45 s. The generated microbubbles were then diluted to 10 mL suspension with PBS, and washed 3 times by centrifugation flotation in a bucket-rotor centrifuge (Model 5804, Eppendorf; Westbury, N.Y.) at 250 G for 5 min. The microbubble cake was finally diluted with PBS for subsequent experiments. For NHS-FITC binding, pH adjusted PBS solution (pH 8.5) was used. PBS at physiological pH (7.4) was used for all other experiments, unless otherwise stated.

Microbubble size isolation was done as described elsewhere.29 This technique allowed us to more effectively isolate microbubbles with a desired diameter due to their multimodal size distribution. Three microbubble size ranges were isolated: 1-2 μm, 4-5 μm and 6-8 μm. An Accusizer optical particle counter (NICOMP Particle Sizing System; Santa Barbara, Calif.) was used to measure the size distribution and particle concentration. Flow cytometry (1×105 events) was performed immediately afterward using an Accuri C6 flow cytometer (Accuri Cytometers Inc.; Ann Arbor, Mich.). The forward-scatter height (FSC-H) threshold was adjusted to delineate the microbubble populations from instrument and sample noise. The system setting was held constant for all subsequent measurements.

For clinical use, the size-selected microbubble cake may be held in a sterile condition in a sterile container such as a bottle or syringe. The container with the cake may be stored for a period of time and maintained ready for use at a suitable temperature that provides a desired shelf life. For example, the microbubble cake can be refrigerated at 10 C. The ideal temperature may depend on the gas inside the bubbles, for example which may determine the solubility of gas in the material making up the shell. By forming a cake, the risk of diffusion of gas out of the particles may be reduced.

Based on the size distribution and concentration data obtained using the Accusizer, each microbubble sample was diluted to about 1×109 #/mL. It is reported that the average projected area per lipid molecule for DSPC is 0.44 nm2.32 Keeping the same value for all other lipid species, the total number of lipid molecules on the shell surface was calculated. Assuming the uptake of lipid molecules to the shell was the same for all species, the relative molar ratio of lipid components in the microbubble shell will be the same as in the bulk suspension.33 The total number of functional groups present on the surface of microbubbles was then calculated, and the excess amount of FITC ligand (molar ratio varied from 0.04:1 to 100:1 and 0.05:1 to 1.5:1 for NHS-FITC:DSPE-PEG2000-A and SA-FITC:DSPE-PEG2000-B, respectively) needed for conjugation was obtained. Samples were incubated with the indicated amount of FITC ligand in the dark overnight on a benchtop rotator at room temperature. Unreacted NHS-FITC or SA-FITC was removed by centrifuging/washing the sample at 250 G for 5 min. The concentrated microbubble cake was then re-suspended to 1×109/mL in PBS and analyzed by flow cytometry.

Microbubble suspensions were incubated with the indicated amount of FITC ligand in the dark on a benchtop rotator at room temperature. FITC ligand binding was continuously monitored for 6 hours. 2 μL samples were taken out at different time points for flow cytometry measurement. A pseudo-first order association kinetics model, given by Equation 1, was used to fit all median fluorescence intensity versus time data,

Y=Yₘₐₓ(1−e^{-k}^{_{obs}}^{t}) (1)

where Yₘₐₓ is maximum median fluorescence intensity (MFI) increase and kobs is the observed binding rate constant in units of hr. Curve fitting parameters for each data set were obtained using the nonlinear regression tool in Prism software (Graph Pad Software, Inc; La Jolla, Calif.). All curves showed reasonable goodness of fit with R2 values approximately 0.92 and above, except for SA-FITC ELA 1-2 μm microbubble sample (discussed below).

According to further examples, the following methods were employed.

Phospholipids were purchased from Avanti Polar Lipids, Inc (Alabaster, Ala.), including 1,2-distearyol-sn-glycero-3-phosphocholine (DSPC), 1,2-distearyol-sn-glycero-3-phosphoethanolamine-N-[methoxy(polyethylene glycol)2000] (DSPE-PEG2000), 1,2-distearyol-sn-glycero-3-phosphoethanolamine-N-[maleimide(polyethylene glycol)2000] (DSPE-PEG2000-M), 1,2-distearyol-sn-glycero-3-phosphoethanolamine-N-[methoxy(polyethylene glycol)3000] (DSPE-PEG3000) and 1,2-distearyol-sn-glycero-3-phosphoethanolamine-N-[methoxy(polyethylene glycol)5000] (DSPE-PEG5000). All phospholipids were dissolved in chloroform (Sigma-Aldrich; St. Louis, Mo.) and stored in the freezer at −20° C. The perfluorobutane gas (PFB, 99 wt % purity) used for microbubble generation was purchased from FluoroMed, LP (Round Rock, Tex.) The RGD peptide (cyclo [Arg-Gly-Asp-D-Phe-Cys], 999% purity) was purchased from Peptides International (Louisville, Ky.) and was dissolved in 3 vol % degassed acetic acid (Sigma-Aldrich). The dissolved RGD peptide was aliquoted into 50-μL volume and stored in nitrogen at −20° C. The L-cysteine was purchased from Sigma-Aldrich and was dissolved in 18 MΩ-cm filtered deionized water (Direct-Q Millipore; Billerica, Mass.). The L-cysteine solution was prepared on each day immediately before use to ensure reactivity.

Human complement-preserved serum was purchased from Valley Biomedical (catalog no HC1004; Winchester, Va.). Serum was thawed once to aliquot into 1-mL eppendorf tubes and stored at −80° C. Anti-human IgG-FITC antibody (catalog no F4512) was purchased from Sigma-Aldrich. Both anti-human albumin-FITC antibody (catalog no CLFAG2140) and anti-human C3/C3b-FITC antibody (catalog no CL2103F) were purchased from Cedarlane (Burlington, N.C.) All antibody solutions were stored at 4° C.

The compositions of microbubble samples for all the experiments performed are listed in Table 1. Microbubbles were generated as described elsewhere. Briefly, the indicated amounts of each phospholipid species were mixed, and the chloroform was evaporated The dried lipid film was hydrated with phosphate buffered saline (PBS) mixture (90 vol % PBS:10 vol % 1,2-propanediol:10 vol % glycerol; Sigma-Aldrich) to a final lipid/surfactant concentration of 1 mg/mL. Fully dispersed lipid suspension was then transferred to a 3-mL serum vial and sealed for headspace PFB gas exchange. Microbubbles were formed by shaking with a VialMix (ImaRx Therapeutics; Tucson, Ariz.) for 45 s. The generated microbubbles were then diluted to 10-mL suspension with PBS, and washed 3 times by centrifugation flotation in a bucket-rotor centrifuge (Model 5804, Eppendorf; Westbury, N.Y.) at 250 G for 5 min. The microbubble cake was then diluted in 5 mM EDTA (pH 65) for subsequent experiments.

An Accusizer optical particle counter (NICOMP Particle Sizing System; Santa Barbara, Calif.) was used to measure the size distribution and particle concentration. The amount of RGD peptide needed was then calculated as previously described. RGD peptide was added to react with maleimide functional groups on the distal end of PEG chains at a molar ratio of 30:1 (RGD:maleimide). The reaction was carried out on a benchtop rotator for 12 hours at 4° C. To ensure there were no unreacted maleimide groups, L-cysteine was added at a molar ratio of 1000:1 (L-cysteine:maleimide) after RGD peptide conjugation. The sample was incubated on a benchtop rotator for 30 min at room temperature. Unreacted RGD peptide was removed by centrifuging the microbubble suspension at 250 G for 4 min RGD peptide conjugation was confirmed using HPLC and MALDI-TOF (data not shown) as reported elsewhere. The concentrated microbubble cake was then re-suspended in PBS and analyzed by Accusizer. The median fluorescence intensity was measured using an Accuri C6 flow cytometer (Accuri Cytometers Inc; Ann Arbor, Mich.). For zeta potential measurement, the washed microbubble cake was re-suspended in pH adjusted PBS solution (pH 72) and analyzed using a Malvern Zetasizer Nano-ZS (Malvern Instrument Ltd; Worcestershire, UK).

Serum aliquots were randomly chosen from each batch to test for complement component C3/C3b activity at different time points throughout the entire immunogenicity study C3/C3b activity was measured using an ELISA kit purchased from Assaypro (catalog no EC2101-1) following the manufacturer's instruction. No serum was re-frozen after ELISA assay to ensure complement activity.

1 mL serum was preheated in a water bath at 37° C. using a digital block heater (VWR; West Chester, Pa.) for at least 20 min. A total of 5×108 RGD-conjugated microbubbles were added to the serum, and the size distribution and microbubble concentration was continuously monitored for 2 hours at 37° C. during both Accusizer and flow cytometer. The same amount of sample (6 μL for Accusizer and 4 μL for flow cytometry) was taken out at different time points for measurement to ensure consistency Incubated samples were vortexed regularly to prevent microbubble aggregation at the top of the serum. Flow cytometry size isolated gating was used for data analysis as previously described.

Direct visual confirmation of microbubble fluorescence was performed within 24 hours after FITC ligand binding. Microbubble samples were taken out of the reaction syringe and imaged at room temperature. Images were captured in epi-fluorescence mode using a high-resolution digital camera and processed with Simple PCI software and ImageJ 1.4 g software (NIH; Washington D.C.).

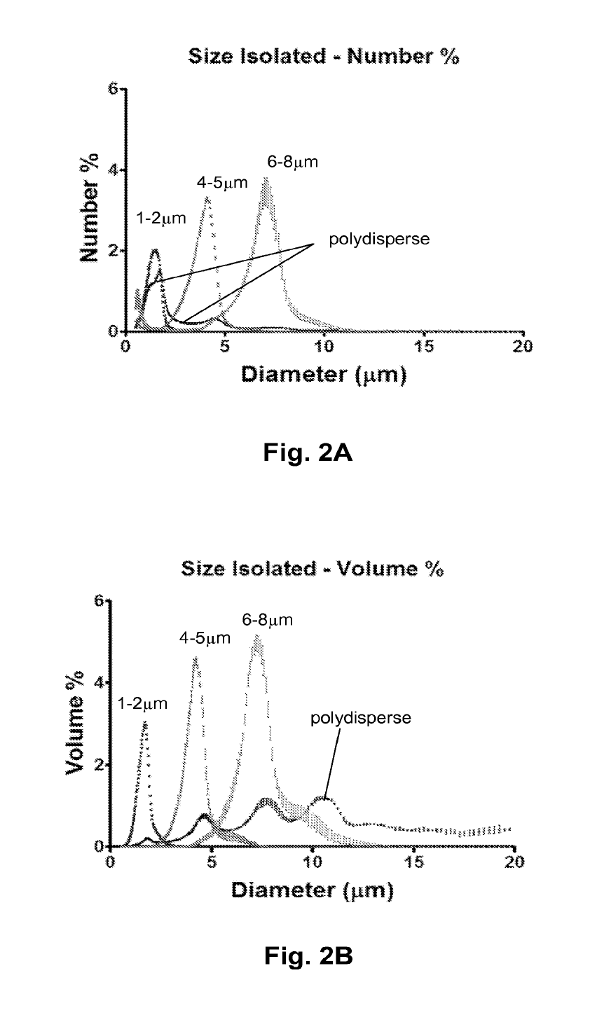

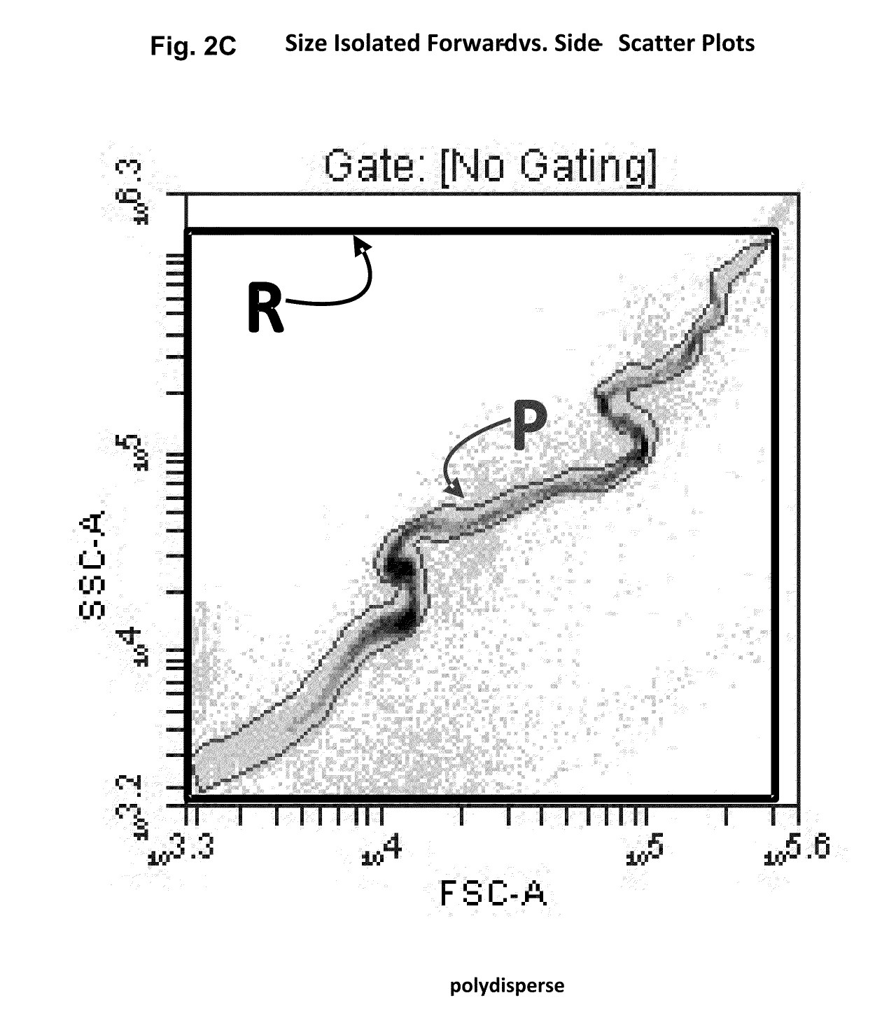

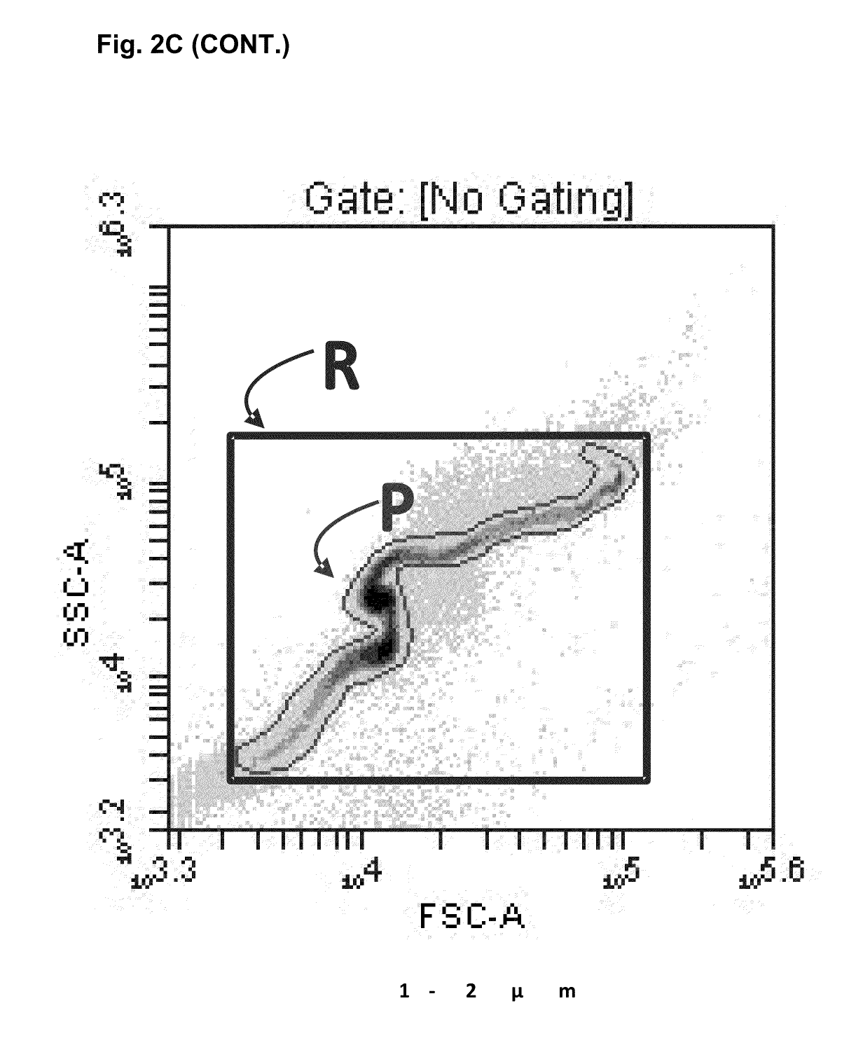

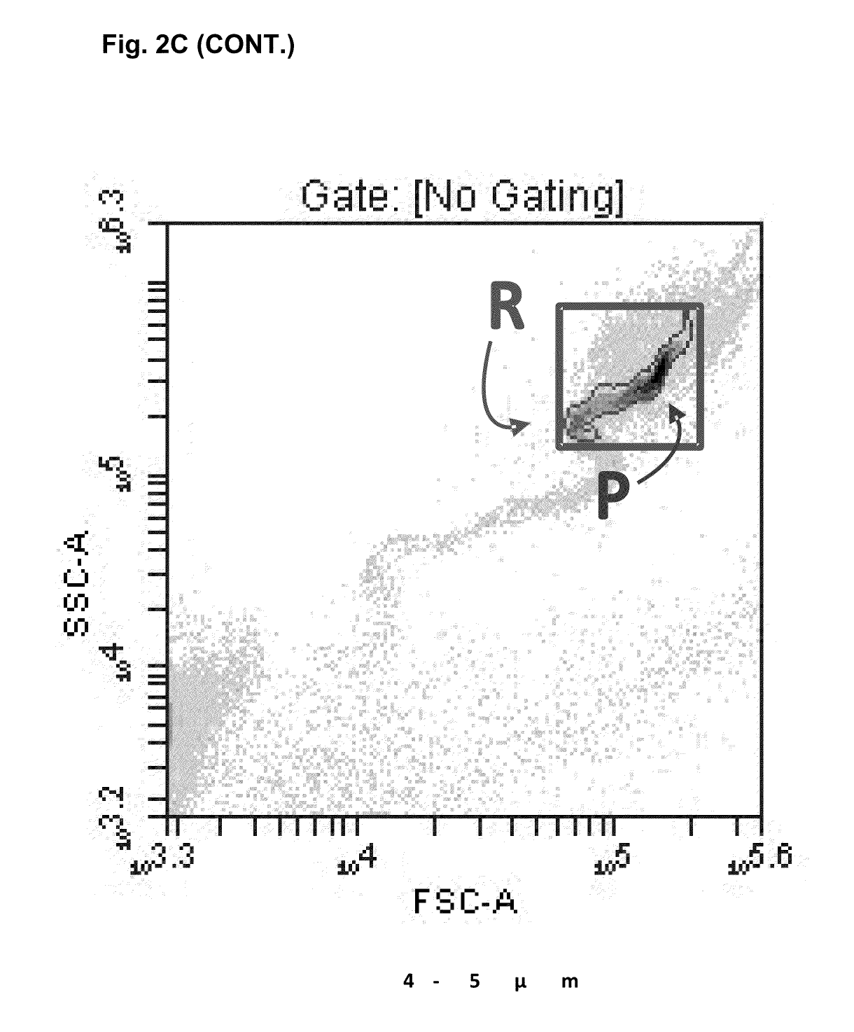

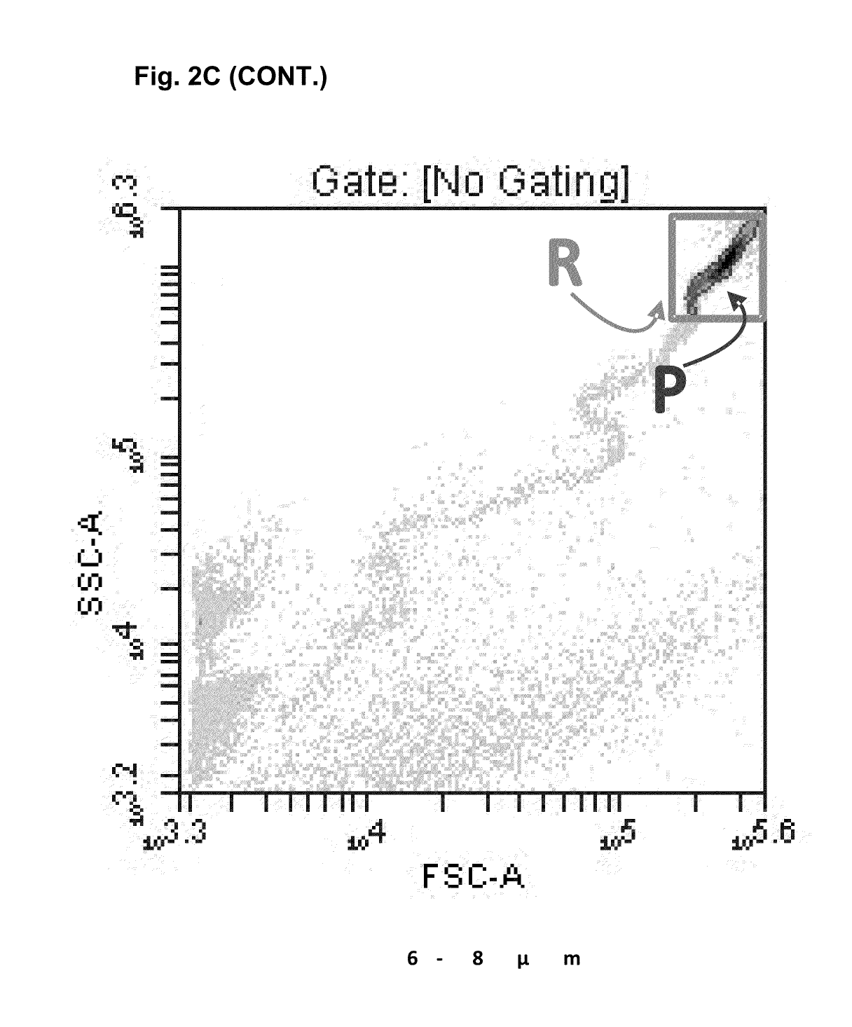

For each set of microbubble components, the vial shaking method produced a milky, white microbubble suspension that was stable over the experimental timeframe. It was shown that small ligands with molecular weight <1 kDa, such as RGD peptides, could diffuse freely through the PEG overbrush and react with functional groups at the distal end of buried PEG chains Here, HPLC and MALDI-TOF were used to ensure the complete attachment of RGD peptides to the surface of BLA microbubbles using the post-labeling technique (data now shown). Since several factors, such as microbubble size and surface charge, could influence the interactions between microbubbles and serum antibodies, the physicochemical properties of the samples were examined (Table 2). Microbubble samples were matched in concentration after the RGD conjugation and/or washing steps. Similar size distributions were measured for all samples, with a dominant peak between 1-2 μm and a secondary peak between 4-5 μm (FIGS. 2A and 2B). The conjugation of RGD peptide to the surface of microbubbles did not affect either the microbubble size distribution or concentration. The number-weighted mean diameters for all microbubble samples were found to be similar, while the volume-weighted mean diameters ranged between 47-82 μm Measurement of zeta potential showed that the negative charge of P2K microbubbles tended to increase by the conjugation of RGD peptide at pH 72. At the same time, the addition of the PEG overbrush (DSPE-PEG5000) into the microbubble shell tended to neutralize this negative charge.

Microbubble size isolation techniques employed differential centrifugation as described elsewhere in publications by Borden. This method provides a rapid and robust method for size selection and reduces polydispersity of microbubble samples. It also isolates microbubbles from precursor liposomes and non-echogenic nanobubbles, which may be recycled for additional microbubble production. Based on the size distribution, it is estimated that only between 1-10% of the original lipid molecules were incorporated into the microbubble shells. A multimodal size distribution was observed using both the Accusizer and the flow cytometer. FIGS. 2A and 2B are the number % and volume % of sizing data from a freshly made (polydisperse) sample. Distinct peaks in both plots were detected, especially in the volume % distribution, indicating dominant size ranges in the microbubble population. These distinct peaks were consistent across all microbubble samples. The corresponding forward-versus side-scatter (FSC vs. SSC) density plot for the polydisperse sample is shown in FIG. 2C. A characteristic serpentine shape was detected. The serpentine scattergram appeared to correlate with the distinct peaks shown in the size distribution determined by Accusizer.

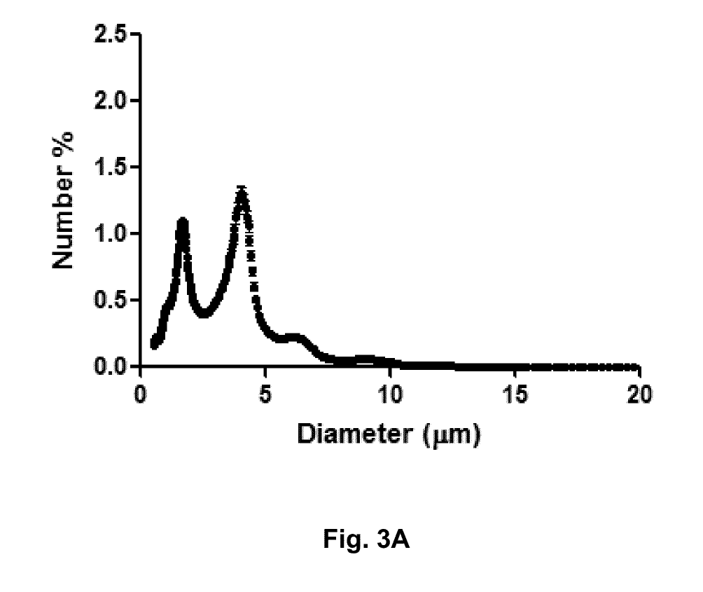

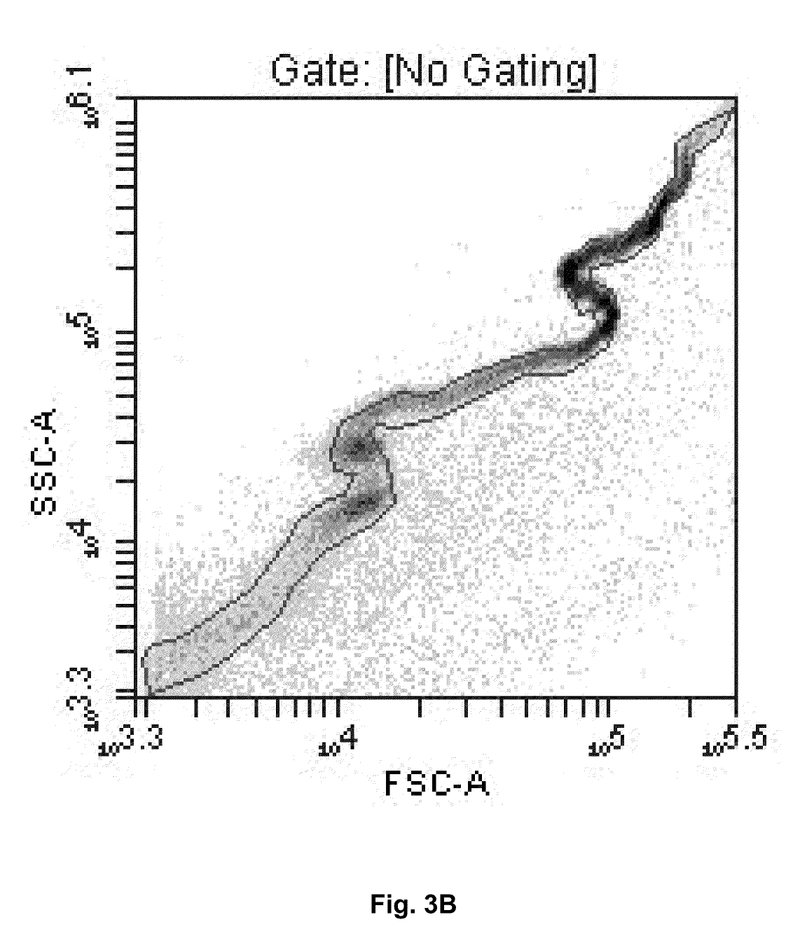

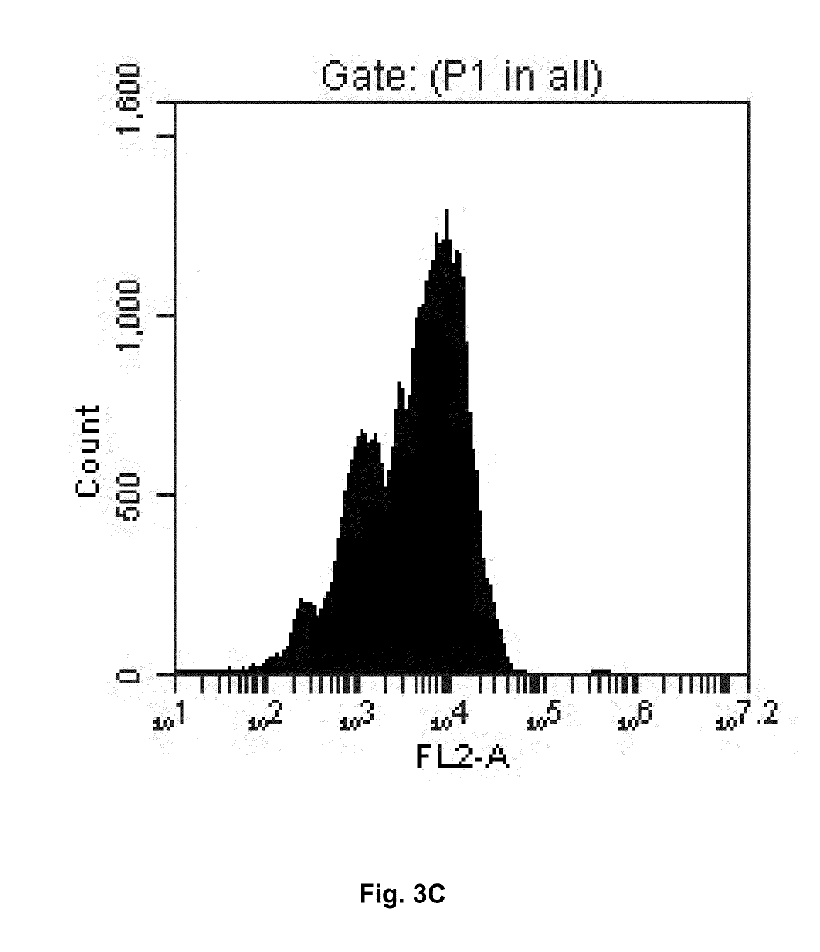

In order to further investigate microbubble size multimodality, a fluorescence-based detection method was employed. DiI labeled microbubbles were centrifuged to remove the liposomes, nanobubbles and some of the 1-2 μm population so that the 1-2 μm and 4-5 μm peaks shown in the number % size distribution were of similar magnitude (FIG. 3A). This step was provided since 1-2 μm microbubbles present in a freshly made suspension dominated the events detected using flow cytometry. Microbubbles with different sizes could not be otherwise detected and represented on the fluorescence histogram. The FSC vs. SSC density plot and fluorescence histogram of the centrifuged sample is shown in FIGS. 3B and 3C. The fluorescence histogram clearly showed a multimodal distribution that corresponded to the Accusizer measurement, lending support to the validity of the multimodal size distribution rather than an optical scattering phenomenon.

FIGS. 2A and 2B also show the sizing data for the size-isolated microbubbles with diameter between 1-2 μm, 4-5 μm and 6-8 μm. The volume-weighted median diameters±standard deviation (SD) were 1.71±0.01 μm, 4.07±0.12 μm and 7.13±0.12 μm, respectively. The corresponding FSC vs. SSC density plots for each population are shown in FIG. 2C. A tight-fitted (P) gate and a rectangular (R) gate was drawn around the densest region of the scatter plots to identify each size subpopulation. The density plots for 4-5 μm and 7-8 μm size-isolated samples showed faint traces of the serpentine shape similar to the polydisperse sample, which might result from the presence of residual smaller microbubbles in the sample. By combining differential centrifugation and flow cytometric gating, it was possible to accurately identify microbubble subpopulations in a polydisperse sample. The gate information determined using the size-isolated samples was saved as a template for all subsequent analyses of polydisperse microbubble suspensions.

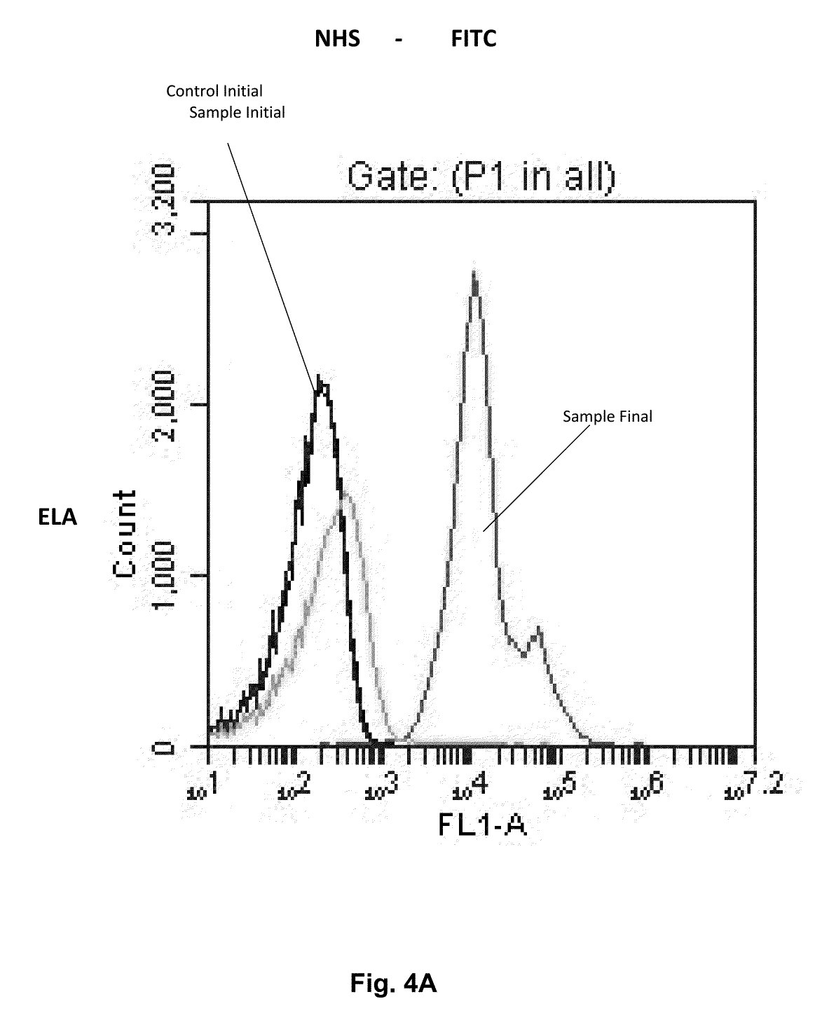

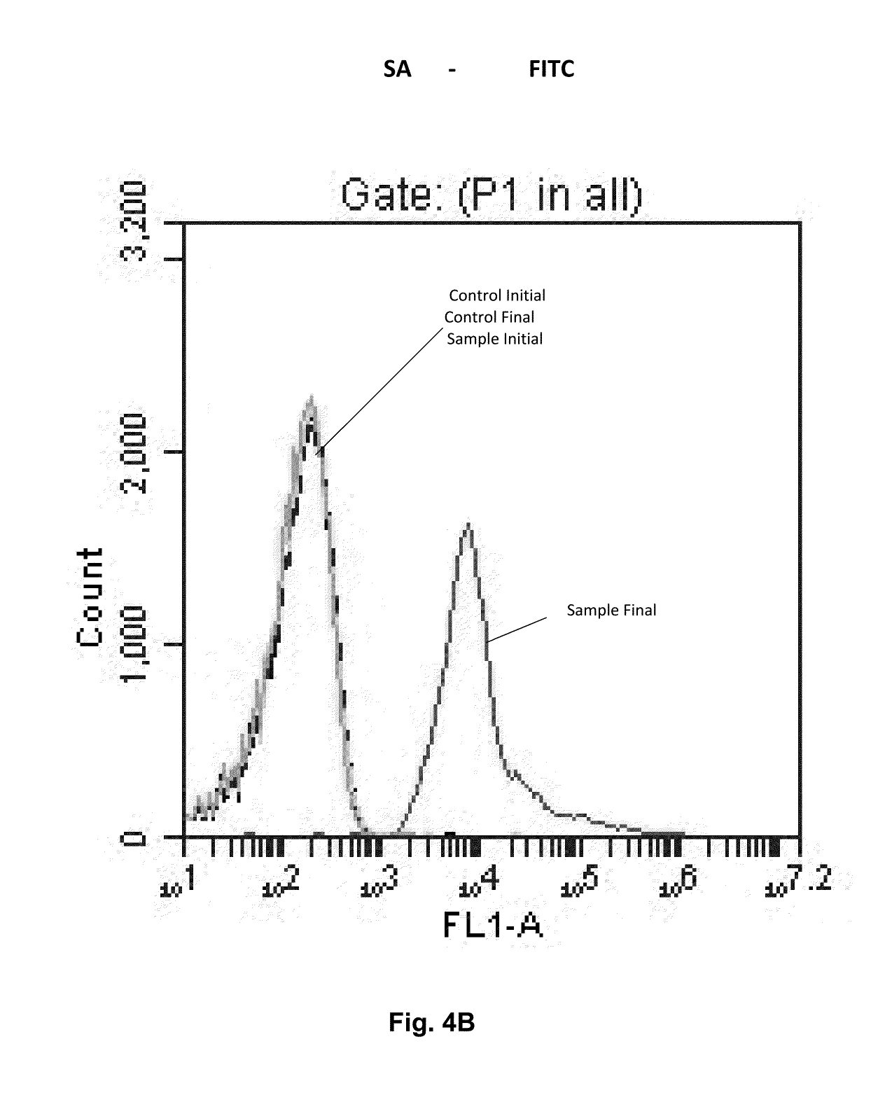

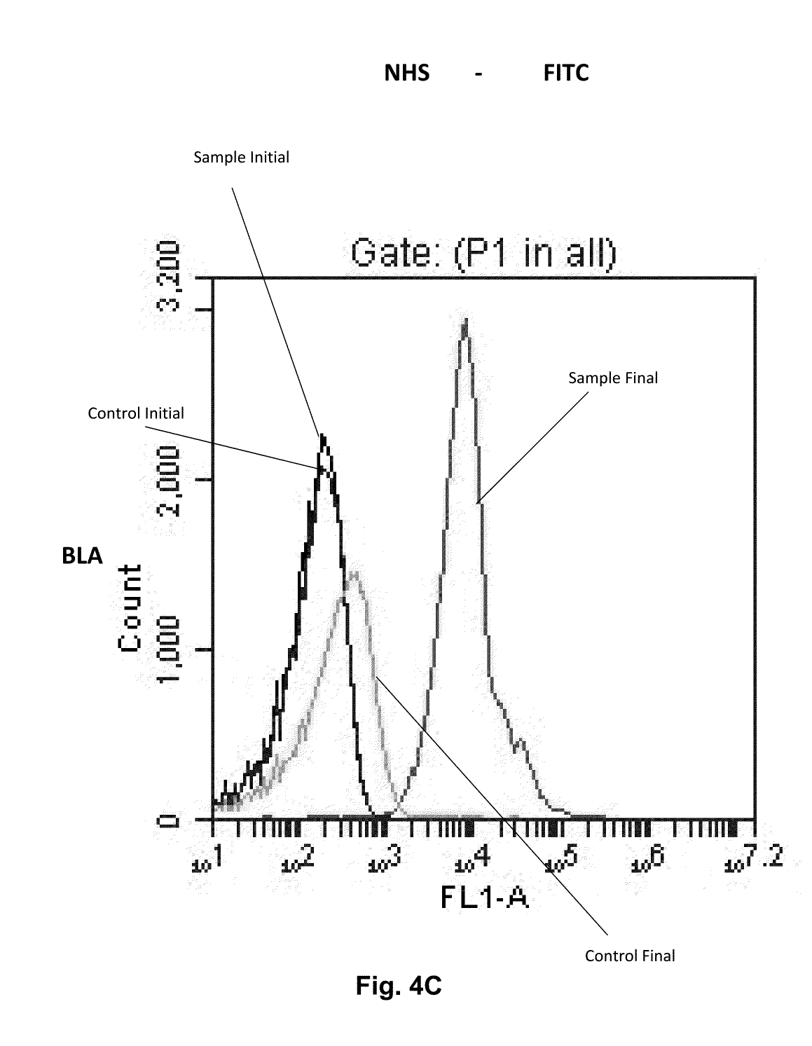

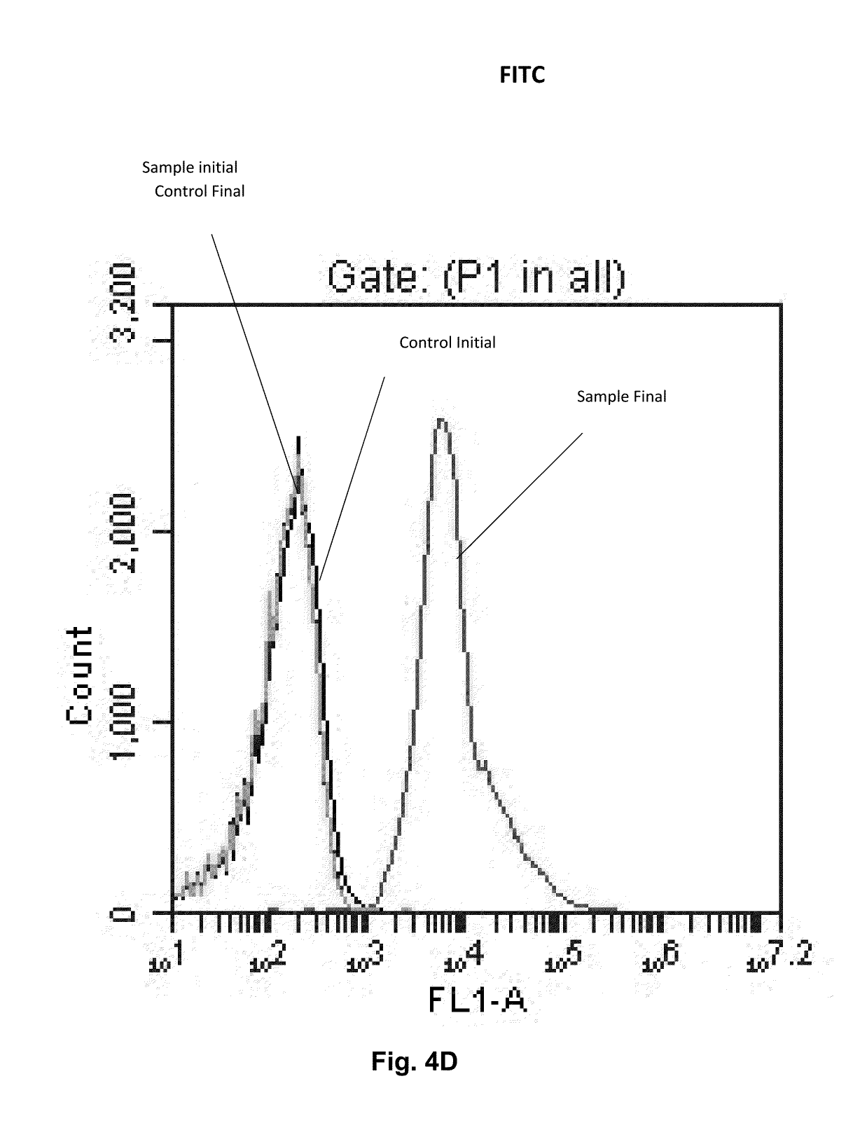

Flow cytometry was used to analyze the binding of the FITC ligands to the PEG-terminal functional groups. FIG. 4 shows sample median fluorescence intensity (MFI) histograms before and after ligand conjugation. The washed polydisperse suspension, which had a dominant 1-2 μm peak and sometimes a relatively large 4-5 μm peak, gave a log-normal or sometimes bimodal distribution in fluorescence intensity (e.g., FIG. 4A). Increased MFI, which was indicated by a shift of the distribution to the right, confirmed the binding of FITC ligand on the microbubble surface. Control microbubbles showed very little or no MFI increase for either NHS-FITC or SA-FITC, regardless of the microbubble surface architecture.

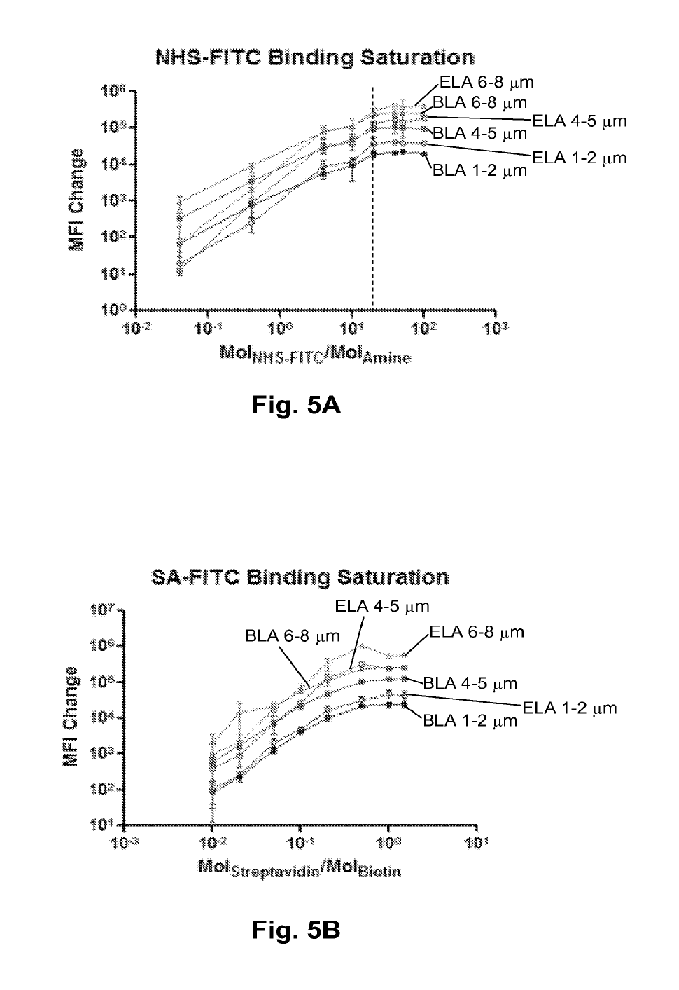

In order to perform a sensitive analysis of ligand conjugation kinetics, studies were done to find the saturation point for NHS-FITC and SA-FITC (FIG. 5). For both ELA and BLA, MFI change increased by about three orders of magnitude when the NHS-FITC:DSPE-PEG2000-A molar ratio was increased from 0.04 to 20 and leveled off to 100 (FIG. 5A). A similar trend was observed for SA-FITC:DSPE-PEG2000-B, although the saturation molar ratio occurred at 0.5 (FIG. 5B). Both molar ratios were identified as the saturation point and used to calculate the appropriate amount of FITC ligand for all subsequent kinetics experiments.

It is commonly accepted that the protective mechanism of PEG polymer chains for liposomes1 or microbubbles2 comes from their flexibility to form a “cloud” that sterically hinders the adsorption of opsonins to the surface, and hence reduces the rapid clearance of these colloidal particles by the reticuloendothelial system (RES).3 It was sought to determine whether this mechanism interferes with the diffusion and attachment of targeting ligands to the buried PEG chains. Smaller molecules, such as NHS-FITC, may be able to diffuse freely through the excluded volume of PEG and bind to the tethered amino groups at the end of the shorter PEG chains for both microbubble surface architectures. Indeed, by monitoring MFI increase using flow cytometry, almost identical NHS-FITC binding kinetics for each size range were obtained between ELA and BLA over 6 hours (FIG. 6). Table 2 summarizes the best-fit values obtained for the pseudo-first order kinetics parameters. Ymax and kobs values for BLA microbubbles were more than 80% of those for ELA independent of microbubble size. This result may confirm that the diffusion and binding of NHS-FITC to the tethered amino groups was not significantly inhibited by the PEG overbrush.

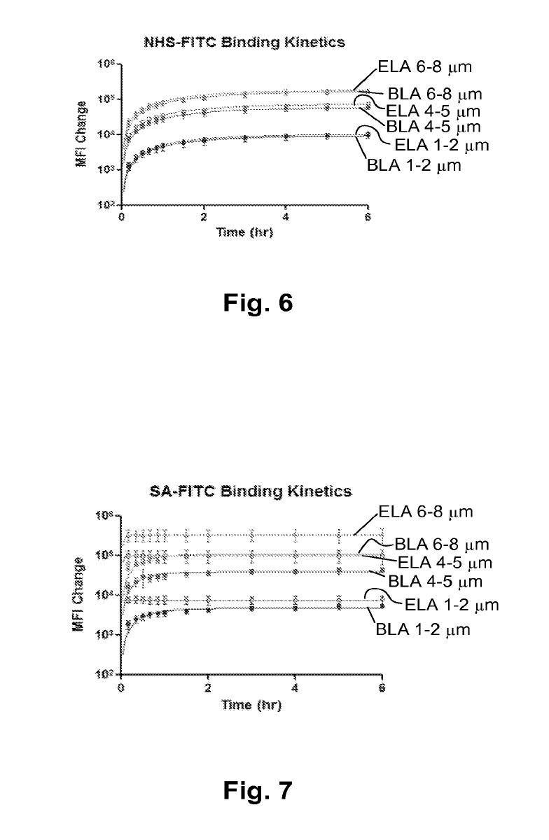

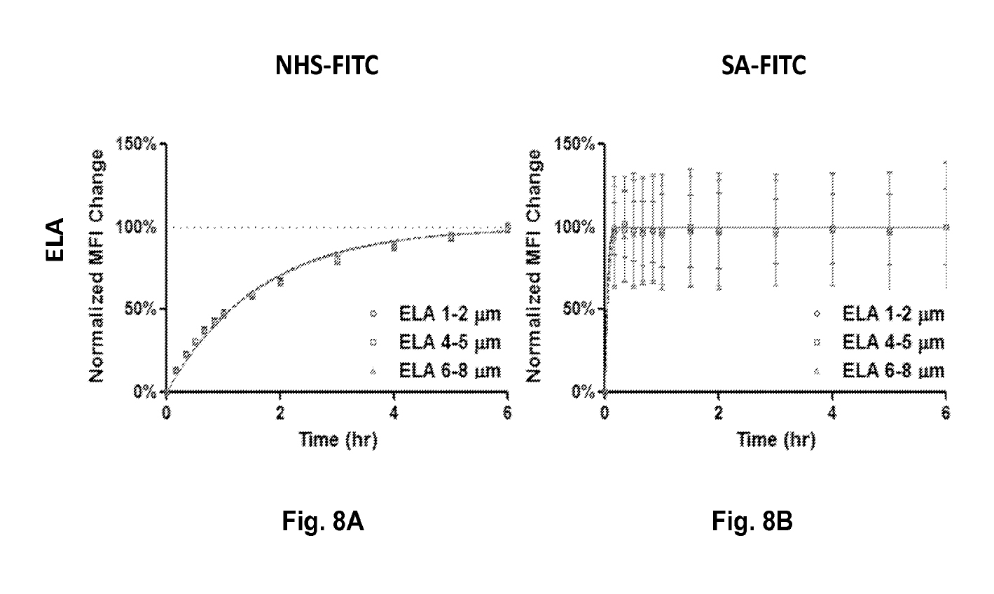

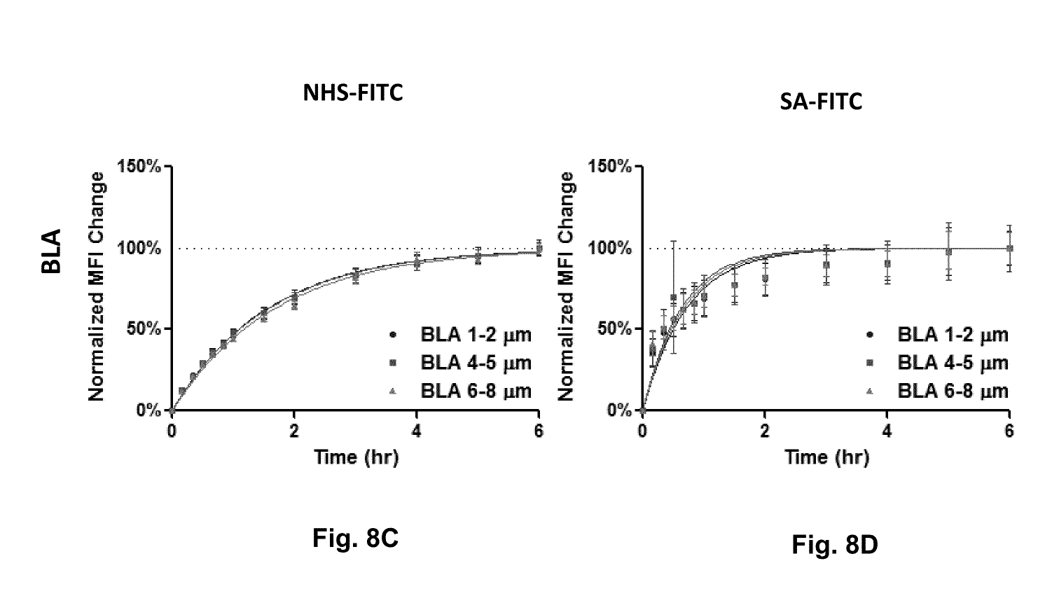

In order to further analyze the binding kinetics, MFI changes were normalized using the final MFI value taken at 6 hours (FIGS. 8A and 8C). Interestingly, binding curves for all three size ranges collapsed to a single curve over the 6-hour experiment. This indicated that NHS-FITC ligand binding occurred at the same rate, independent of microbubble size, in both ELA and BLA designs.

NHS-FITC is similar in molecular weight to several small-molecule peptide ligands, such as cyclic-arginine-glycine-asparagine (RGD). RGD has been shown to bind to an overexpressed angiogenic biomarker, αvβ3 integrin, with high affinity and specificity.34, 35 Using RGD labeled microbubbles with ultrasound molecular imaging, one can monitor and guide therapy of vascular endothelial growth factor (VEGF)-blockage for cancer therapy.36, 37 By showing that NHS-FITC was able to diffuse through the PEG overbrush and bind to the tethered PEG, the feasibility was demonstrated of post-labeling for small-molecule ligands (<1 kDa) to BLA microbubbles to generate stealth targeted ultrasound contrast agents.

It was determined that macromolecules (>10 kDa), such as SA-FITC, could not be able to diffuse freely through the PEG overbrush and bind to tethered biotin end groups due to steric hindrance in the buried-ligand architecture. Indeed, FIG. 7 shows significant differences in SA-FITC binding curves between ELA and BLA for each microbubble size range. ELA binding curves reached the saturation MFI values within the first 10 min of reaction and stayed constant throughout the rest of the experiment. BLA binding curves, on the other hand, showed a gradual increase during the first hour of reaction, reaching the saturation MFI values at approximately 2 hours. The final MFI values for each size range in ELA were significantly higher than those in BLA, with the highest Ymax percentage being only 62% of that for ELA (Table 3, 1-2 μm), indicating that the binding of SA-FITC to buried biotin groups was significantly inhibited by the longer PEG chains.

Again, the effect of microbubble size was investigated. Normalized MFI change showed all binding rates collapsed into a single curve, indicating that SA-FITC binding for microbubbles with different diameters was the same (FIGS. 8B and 8D). Similar to NHS-FITC, the diffusion and binding of SA-FITC was not affected by microbubble size for either BLA or ELA designs.

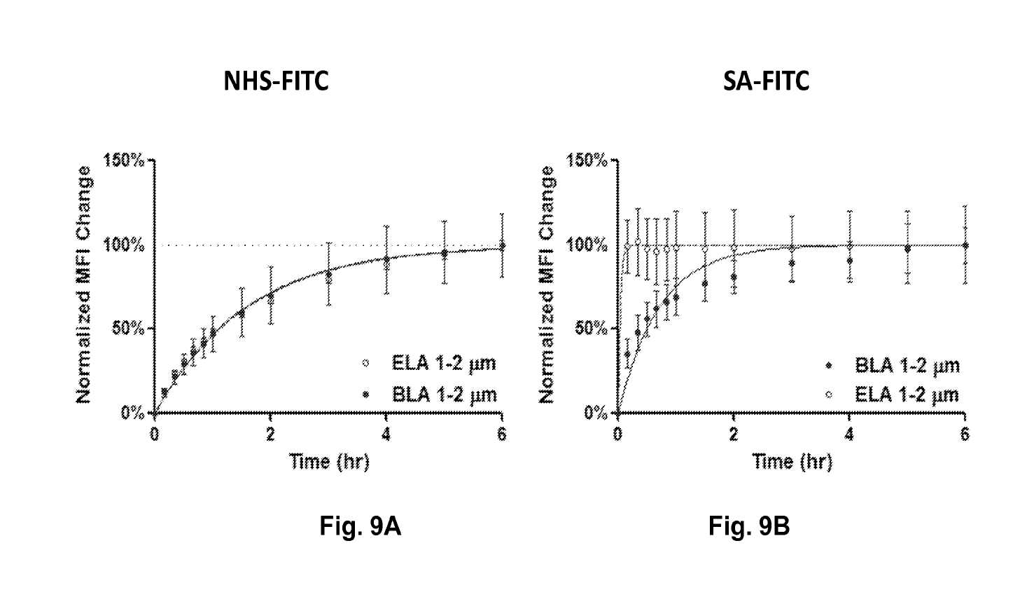

The kinetic curves for the 1-2 μm size range are shown in FIG. 9 to further illustrate the significant difference in binding rate between ELA and BLA microbubbles for different ligand sizes. For NHS-FITC, the normalized binding curves showed very little difference between the two brush architectures over 6 hours, with the fitted kobs values in close agreement between ELA and BLA (Table 4). On the other hand, for SA-FITC binding, the ELA kinetic curve closely resembles a step function with the MFI value reaching its saturation Ymax within 10 min; yet the BLA kinetic curve showed a more gradual increase over time with a much slower binding rate constant. Unfortunately, due to the fact that ELA samples reached their saturation MFI before the first flow cytometry measurement was taken, reliable kobs values were not obtained for a more quantitative comparison.

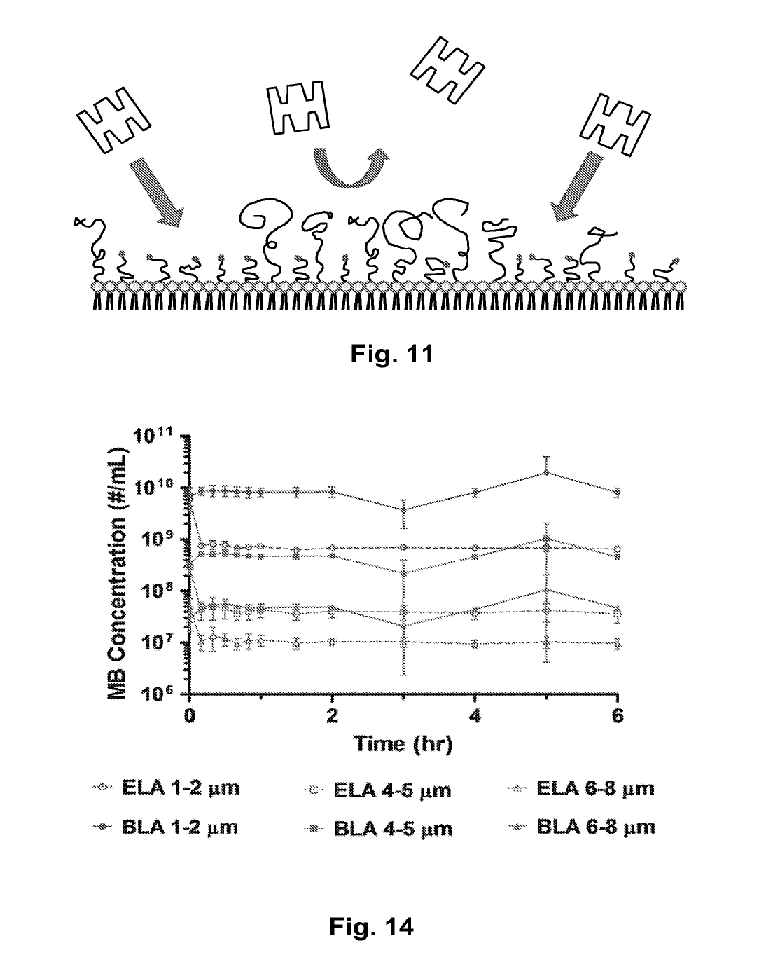

Interestingly, the PEG overbrush did not completely eliminate macromolecule conjugation. Previous findings have shown that phase separation between phospholipid species can exist on lipid monolayers coating the microbubble surface. Two distributions of PEG-lipid conjugates may exist on the surface of microbubbles, which are formed during the initial self-assembly process: domains with DSPE-PEG2000-X and DSPE-PEG5000 well mixed, and peripheral regions comprising mainly DSPE-PEG2000-X (where X=either Amino or Biotin) (FIG. 11). Smaller ligands, such as NHS-FITC, may diffuse freely through the excluded volume of PEG and bind to the amino groups in the center of the domains. Needham et al. 4, 16 modeled the diffusion of small molecules through a surface-grafted PEG layer as a function of polymer molecular weight. It was found that as the PEG molecular weight increased, the volume fraction of polymer in the excluded volume decreased, indicating that there was more free-water volume for small molecules to penetrate. The results demonstrate that the presence of the DSPE-PEG5000 overbrush does not interfere with the diffusion of NHS-FITC molecules to the tethered amino groups.

In contrast, the binding of SA-FITC to tethered biotin groups in BLA microbubbles was significantly lower (˜45%) than that for ELA microbubbles. This may be because the observed binding of SA-FITC mainly occurred to the tethered biotin groups on the peripheral DSPE-PEG2000-B chains, where the longer DSPE-PEG5000 chains did not form a complete dense cloud over the buried biotin groups. In the central domain regions, SA-FITC molecules could not overcome the steric hindrances of DSPE-PEG5000 chains, and therefore were physically prevented from binding to the biotin groups, which resulted in the differences in the final saturated MFI values between ELA and BLA microbubbles.

It is also possible that the transient excursion of PEG chains could result in some SA-FITC:DSPE-PEG2000-B binding in the central regions of the domains. If this were the case, SA-FITC would continue to bind, and a linear increase of MFI over time would be observed. However, the significantly lower MFI values of BLA microbubbles and the non-linear binding curves both indicated that such events occurred at a very low frequency over the experimental period, and it was the physical inhibition of SA-FITC molecules due to the steric repulsion that resulted the difference in final MFI values between BLA and ELA microbubbles.

Using a surface force apparatus, Moore et al.13 measured the specific and nonspecific forces between a streptavidin-coated surface and a bimodal PEG mushroom with buried biotin with similar lipid composition as presented here. It was found that the presence of longer PEG did not significantly change the capture distance of specific adhesion even though the steric repulsion between these two surfaces was increased. The discrepancy between their results and ours can be explained by the differences in experimental design. Moore et al. had two surfaces slowly approach each other, allowing the tethered biotin end groups enough time to equilibrate and bind to apposed streptavidin molecules under compression. The study is different in that microbubbles and ligand molecules diffused freely in solution throughout the reaction. There were no external forces acting on the system. Therefore all measured binding events were the result of passive diffusion. It was shown that BLA microbubbles were able to successfully bury the targeting ligands and reduce specific adhesion in comparison with ELA microbubbles when no USRF was applied. In the current study, the presence of longer PEG showed a significant effect on the binding of SA-FITC, indicating that the diffusion of macromolecules through the PEG overbrush was partially inhibited.

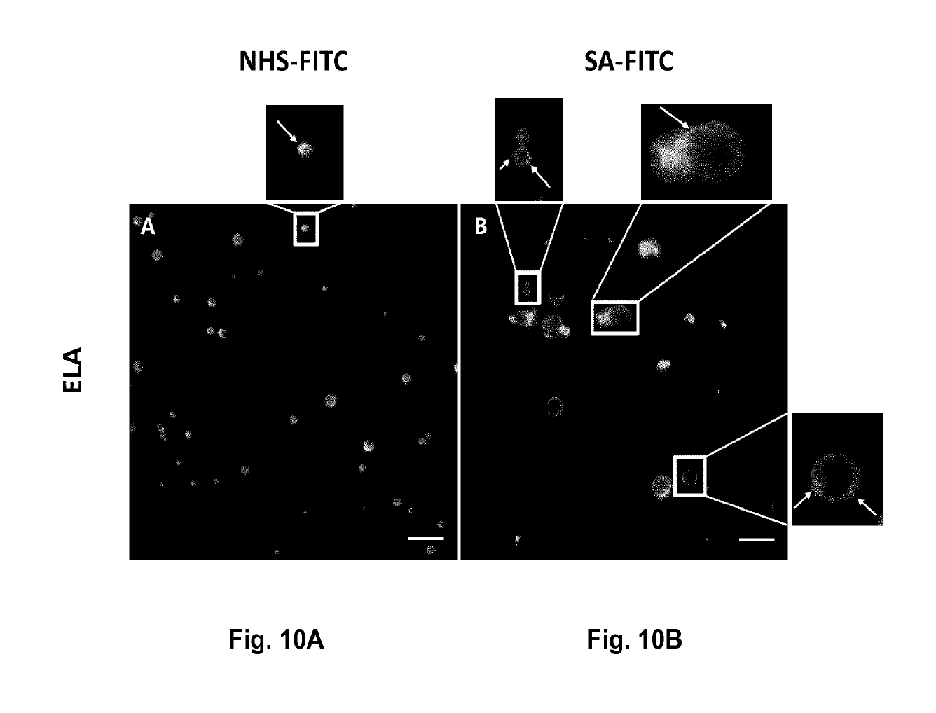

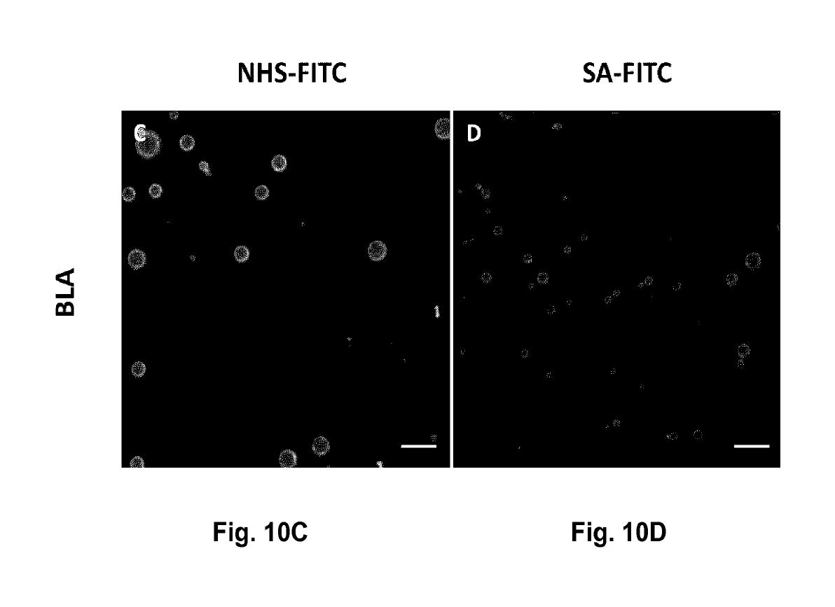

Epi-fluorescence microscopy images provided direct visual confirmation for the conjugation of FITC ligands to the surface of microbubbles (FIG. 10). All microbubble samples appeared to be stable during observation. When compared with bright-field images, all polydisperse microbubbles were visible under epi-fluorescence mode (data not shown). There was no preferential attachment of ligands due to microbubble size. Microstructural features within the lipid shells were detected (see arrows in FIGS. 10A and 10B), indicating non-uniform distribution of FITC ligand on the microbubble surface.

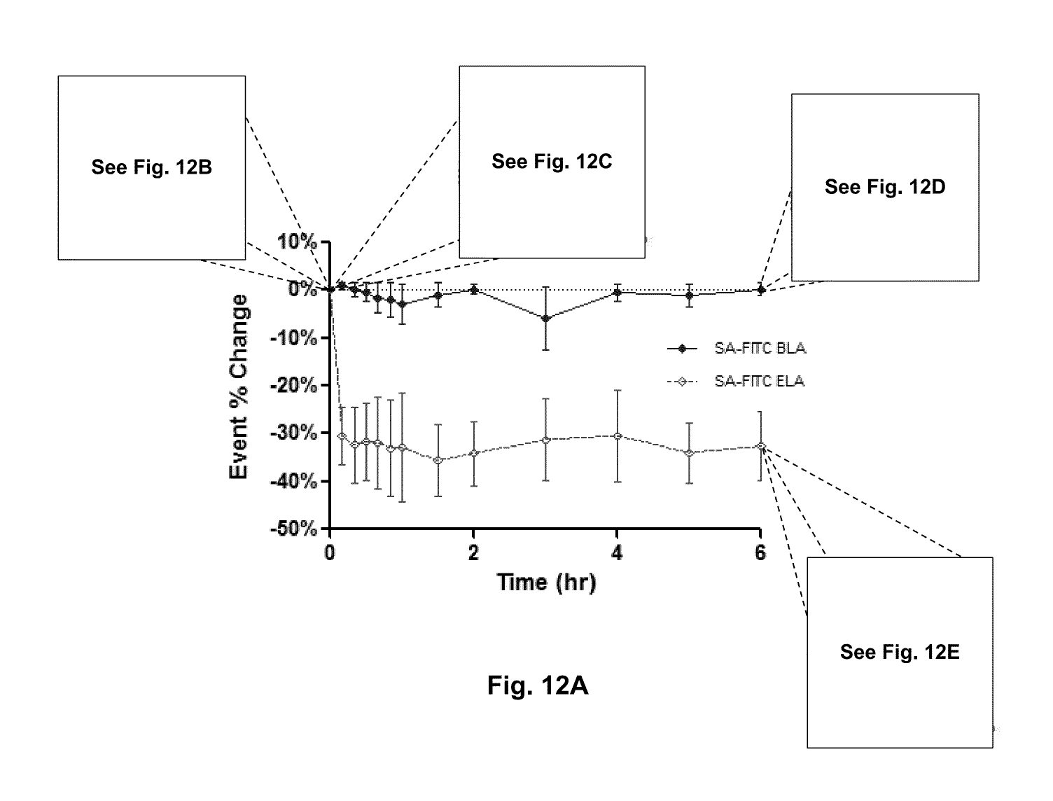

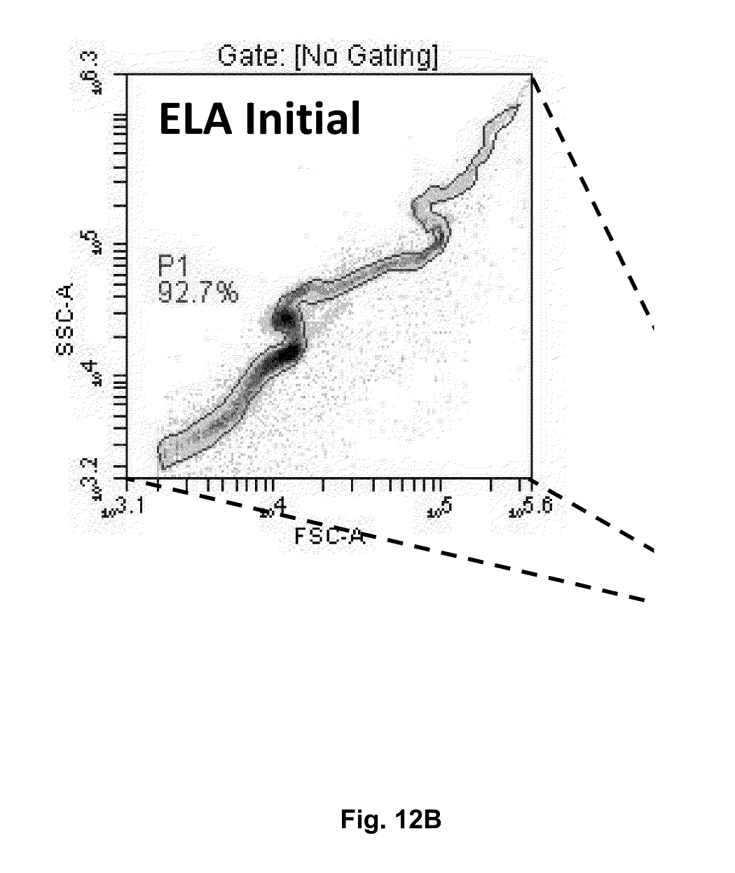

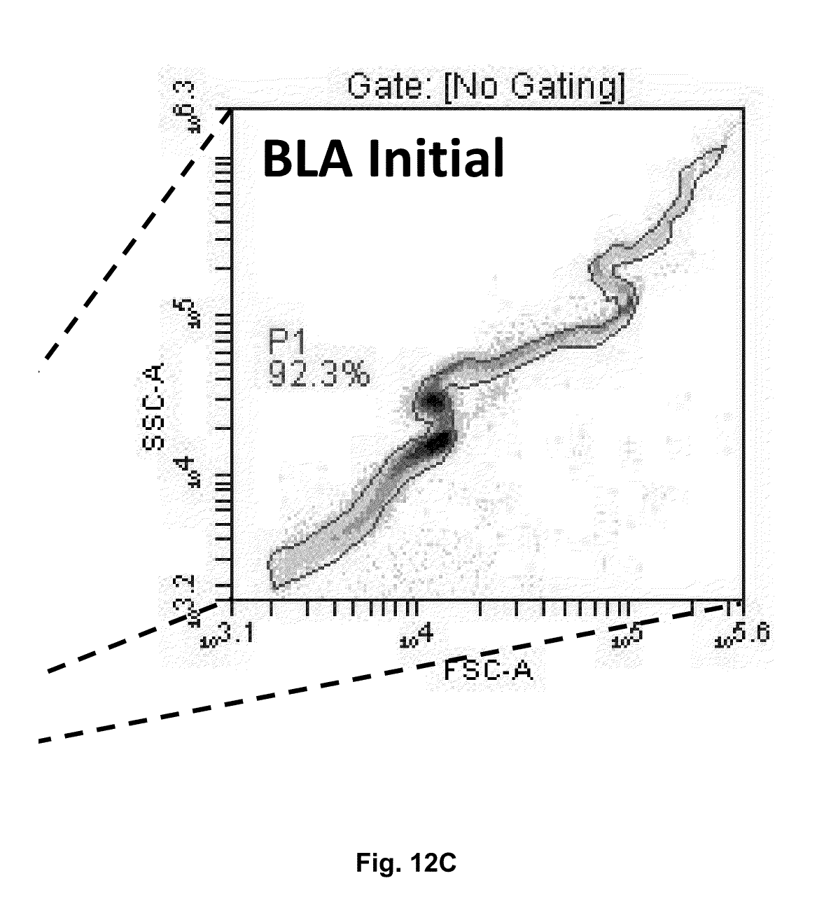

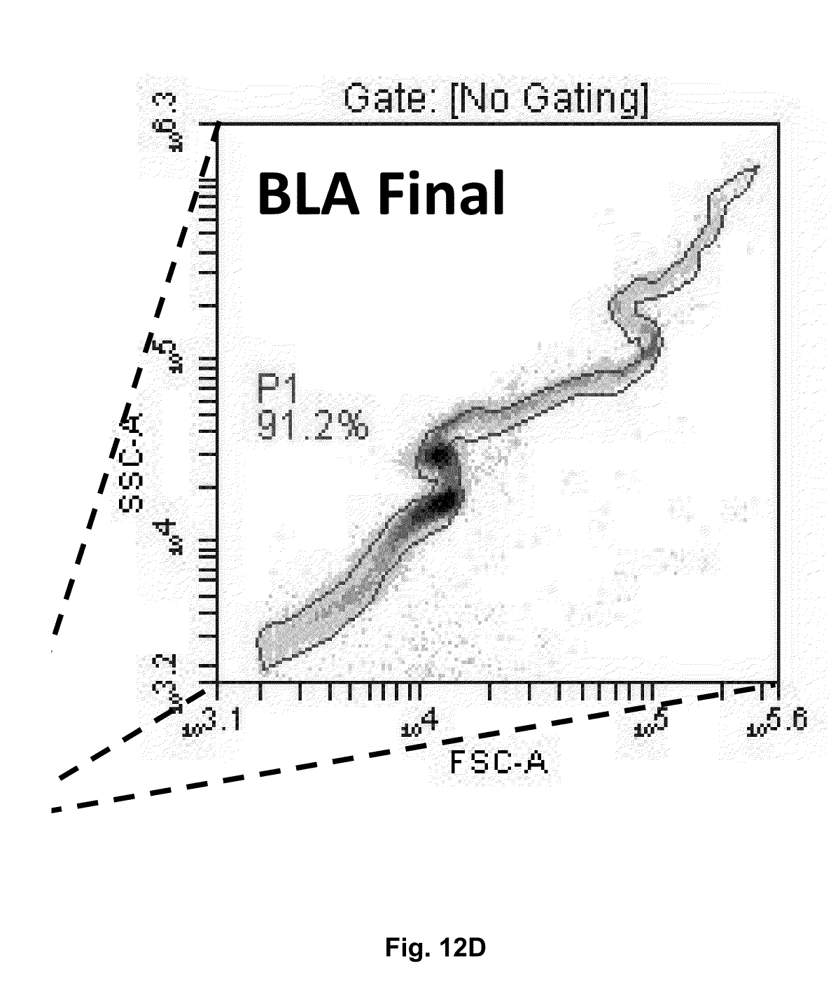

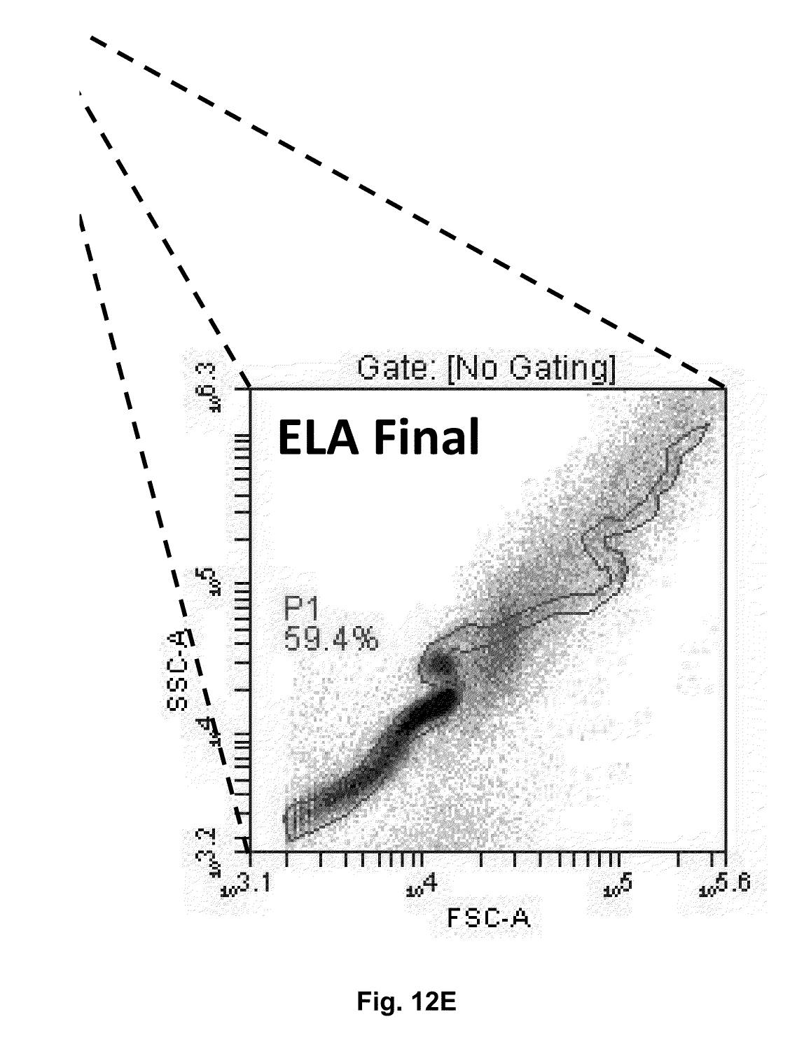

During the preliminary screening for ligand saturation experiment, it was observed that the number of events (event %) that fell within the polydisperse P gate on the FSC vs. SSC plot stayed relatively constant for all microbubble samples except for SA-FITC:ELA binding. A significant decrease of event % inside the gate was repeatedly observed immediately after SA-FITC binding to ELA microbubbles, and the relative change stayed constant throughout the 6-hour experiment (FIG. 12). Surprisingly, this change of event percentage was not accompanied by a corresponding microbubble concentration decrease (data not shown). Upon further investigation, such phenomenon could also be detected using the tight-fitted size-isolated P gates, and the percentage change correlated to the total number of microbubbles of each size range presented in the polydisperse population. However, when using the rectangular R gates, no change of event % was detected. It was then proposed that this change of scattering property resulted from a change of microbubble surface structure. Epi-fluorescence Z-scan images were taken in order to verify this proposal. FIG. 10B (upper right) shows typical SA-FITC labeled ELA microbubbles exhibiting complex surface structure (e.g. folds, domains and protrusions) that were not normally found on either SA-FITC labeled BLA microbubbles or NHS-FITC labeled ELA microbubbles. Similar structures were found on almost all SA-FITC bound ELA microbubbles that were large enough (diameter >˜3 μm) to be examined by optical microscopy. No visible changes, such as collapse aggregates forming or vesicles shedding, were observed for these microbubbles over the observation time period (typically around 10-15 min).

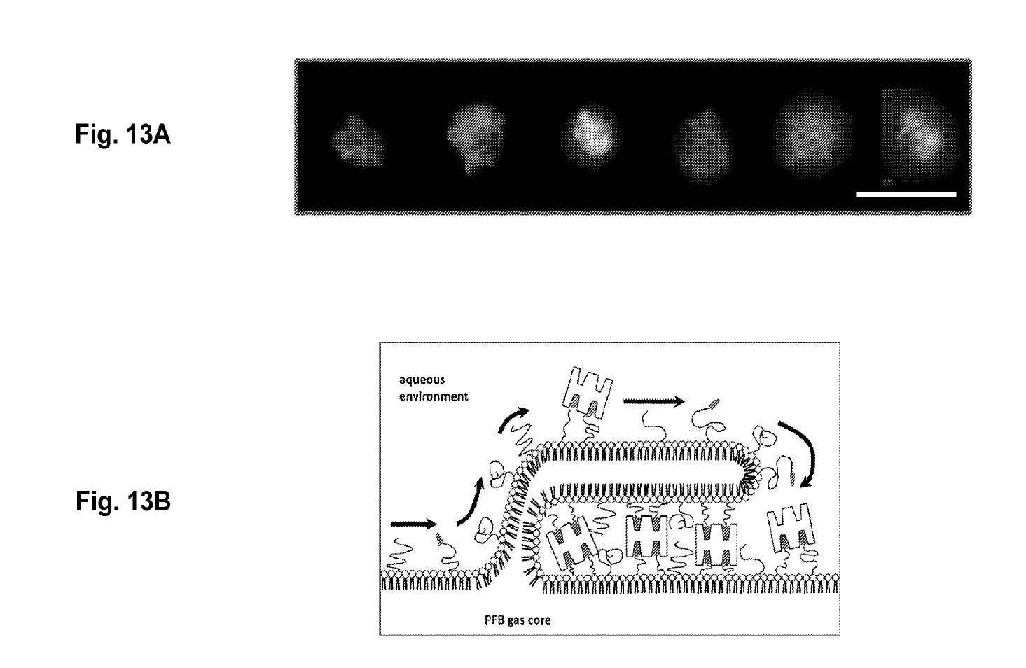

The streptavidin-induced surface structures (folds and protrusions) were not observed for either SA-FITC labeled BLA microbubbles or NHS-FITC labeled ELA microbubbles, and can be correlated to the flow cytometry measurement very closely (FIG. 12). FIG. 13B shows a cartoon concept of the streptavidin-induced surface structure. An explanation may be that incomplete surface coverage of biotin moieties by the SA-FITC lead to cross-linking between the monolayer shell and folds extending into the aqueous phase. (Small bilayer folds and protrusions are likely present as defects in the microbubble shell prior to SA-FITC binding.) The cross-linking may lead to an effective spreading pressure of the bilayer fold over the monolayer surface, which pulled more lipid from the monolayer plane and into the fold. As the spreading proceeded, a fold would grow until the SA-FITC molecules, which were the limiting reagent, were depleted, or SA-FITC molecules reached their maximum surface density. Fold nucleation and growth would likely be guided by shell heterogeneities and would induce gas dissolution from the core, consistent with results presented in FIGS. 10 and 12. This explanation was inspired by previous work by others showing adhesion between apposing lipid vesicles induced by avidin-biotin interactions. The biotin-avidin spreading pressure was able to bend and stretch the bilayer membranes to form large contact regions (e.g., plaques). Previous work done by Nam and Santore47 showed that the time scale for spreading in generally on the order of seconds. For the experiments, by the time the first data point was taken at 15 minutes, it is assumed that the growth and spreading of the folds and protrusions were completed, and the system reached a new equilibrium state.

In order to test the hypothesis that the formation of these surface structures was a streptavidin-biotin mediated phenomenon rather than a manifestation of gas core dissolution owing to dilution, the microbubble concentration change over the 6-hour experiment period were plotted for each size range using the flow cytometry data (FIG. 14). For ELA microbubbles binding to SA-FITC, all size ranges showed a significant decrease in concentration within the first 10 min and stayed constant throughout the rest of the experiment. For BLA microbubbles, on the other hand, none of the size ranges showed a significant change of concentration. Since both samples were prepared and diluted in the same manner, this result allowed us to rule out the possibility that the observed surface structures resulted simply from microbubble dilution.

The absence of the wrinkled structure observed for BLA microbubbles could be due to inhibited macromolecule diffusion into the bimodal PEG layer. The availability of the tethered biotin group in a bimodal PEG brush was much lower than for the ELA counterpart. If there were not enough biotin-avidin interactions within a close proximity, the system may not have been able to bend and stretch the monolayer enough to promote fold growth.

Mechanical pressurization may be used to create wrinkled microbubbles with increased surface area for loading targeting ligands and facilitating adhesion. +++Another method for inducing complex surface structure formation that resulted similar folds and protrusions. More importantly, it was shown that these surface structures could be quantified using flow cytometry. However, whether these SA-FITC labeled ELA microbubbles can also stabilize specific adhesion is still unknown.

NHS-FITC and SA-FITC were used as model molecules to post-label exposed-ligand architecture (ELA) and buried-ligand architecture (BLA) microbubbles. For small molecules, such as NHS-FITC, the diffusion and binding to the tethered amino end groups were not affected by the PEG overbrush in BLA microbubbles, and the overall binding rate between ELA and BLA microbubbles were the same. On the other hand, for larger molecules, such as SA-FITC, the diffusion and binding to the tethered biotin end groups was partially prevented by the PEG overbrush due to steric hindrances for BLA microbubbles, and the binding rate was significantly reduced. The total binding capacity for BLA microbubbles was significantly lower for macromolecules in comparison to ELA microbubbles (˜40%), suggesting a possible phase separation between lipopolymer species on the surface. These results indicate the that small molecules may diffuse through the excluded volume of PEG chains and react with surface functional groups, while larger molecules were significantly impaired. Post-labeling with BLA microbubbles is therefore highly feasible for small ligands (<1 kDa) for generating targeted ultrasound contrast agents. It has been shown that ligand conjugation was not affected by microbubble diameter regardless of the ligand size. For both small and large ligands, the binding kinetics for all microbubble size classes was the same over the experimental time period. Complex surface structures, or wrinkles, may result through streptavidin conjugation to ELA microbubbles. The tight serpentine shape P gate and the event % parameter can be used together with epi-fluorescence microscopy to detect these surface structures. Flow cytometry can give a quick quantitative indication of the percent of microbubbles in a given suspension that deviate from the normal spherical shape, while microscopy offers direct visual confirmation of the surface structure.

The following describes further methods and analysis for the further examples.

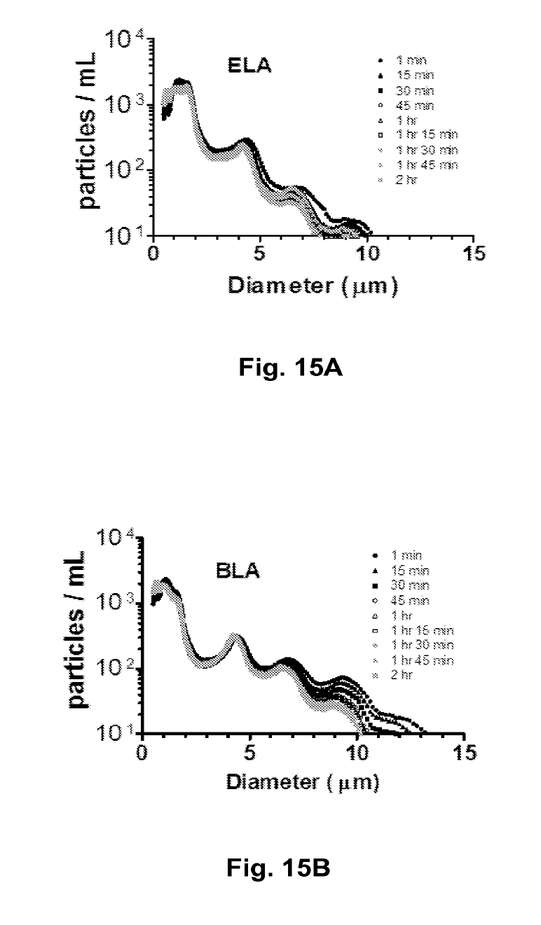

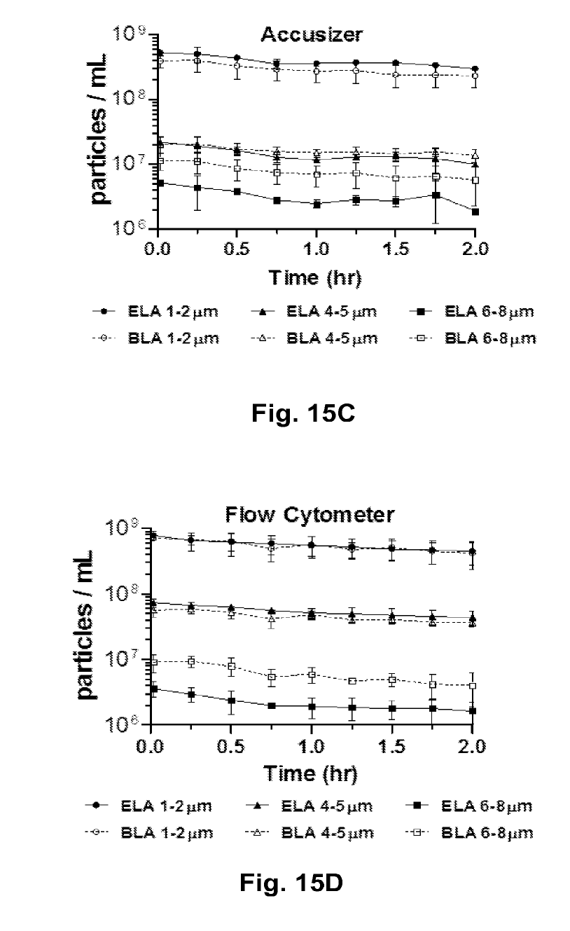

The stability of microbubbles with 5% RGD peptide during incubation in human serum at physiological temperature was investigated FIGS. 15A and 15B shows the size distribution change for ELA and BLA microbubbles, respectively, during the 2-hour incubation as measured by the Accusizer. For both surface architectures, smaller microbubbles with diameter less than 2 μm showed a decrease in number detected over time, while larger microbubbles showed no significant change. The total microbubble concentration change was monitored using both the Accusizer and the flow cytometer (FIGS. 15C and 15D). Both detection methods showed results that were in good agreement a decrease in microbubble concentration was observed for both surface architectures at all size ranges at the end of 2 hours, with the highest decrease being 59% and 53% for ELA and BLA 6-8 μm microbubbles, respectively. However, the concentration decrease for all microbubble samples after 30 min incubation ranged from only 11% to 30%. Targeted microbubbles, regardless of surface architecture, were stable in human serum during incubation at physiological temperature within the time scale for a typical ultrasound contrast imaging session (˜30 min).

Undiluted complement-preserved human serum was used for all experiments. To ensure the validity of the immunogenicity data, complement activity of the serum samples was continuously monitored by measuring complement component C3/C3b activity of randomly chosen serum aliquots throughout the study FIG. 16 shows the quantified C3/C3b activity as measured by ELISA assay for all 38 samples. The measured C3/C3b activity was 30±16 μg/mL of serum (mean±SD). The human serum samples from different batches were statistically identical in terms of complement C3/C3b activity, and the aliquots were stable for the duration of the experiments, By showing consistent complement activity, it was ensured that any measured C3/C3b binding difference in the immunogenicity study was due to the difference in complement activation by various microbubble samples, not due to batch-to-batch variability in serum C3/C3b activity.

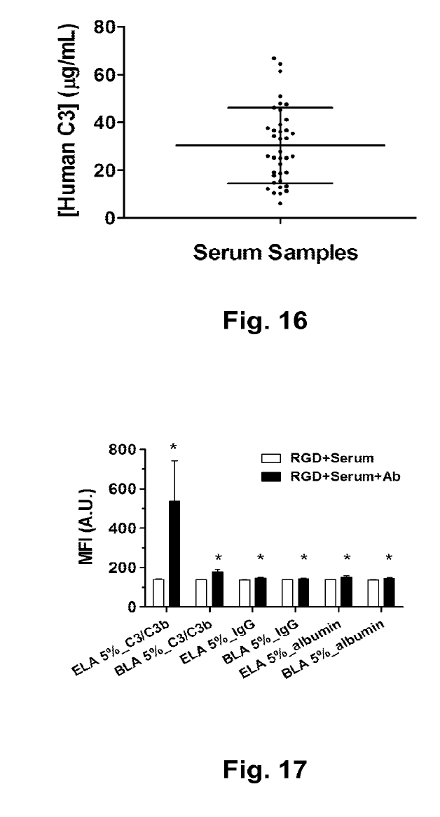

The binding of human complement component C3/C3b, IgG and albumin to targeted microbubbles with 5% RGD conjugated to the surface was examined. Sufficient incubation time (2 hours) was given to allow the full exposure of microbubbles to the serum environment, Detection of fluorescent antibodies by flow cytometry allowed an assessment of serum factor binding to the microbubble shells. FIG. 17 shows the median fluorescence intensity (MFI) values for 1-2 μm ELA and BLA microbubbles after incubation with human serum and FITC-antibodies. The 1-2 μm size range was chosen because these microbubbles were the most abundant in all the samples and could correctly represent the MFI trend for the entire population. All three serum factors were detected on the targeted microbubble samples. However, increases in MFI for IgG and albumin were small (<10%) compared to C3/C3b binding to ELA and BLA microbubbles (38-fold and 13-fold increase, respectively). This indicated that complement C3/C3b was the main opsonin involved with the recognition of targeted microbubbles by the immune system.

Epi-fluorescence microscopy images provided direct visual confirmation of FITC-antibody binding to the surface of targeted microbubbles. Only anti-human C3/C3b FITC-antibody labeled targeted ELA microbubbles were visible under epi-fluorescence mode. FIGS. 18A and 18B show both the bright field and epi-fluorescence images for the same field of view of these polydisperse microbubbles. All microbubbles appeared to be stable during observation. No visible changes, such as collapse, aggregate formation or vesiculation were observed for these microbubbles over the observation time period (typically around 10-15 min). Almost all microbubbles visible under the bright field mode were also seen under the epifluorescence mode, indicating FITC-antibody binding to the surface. There was no preferential binding due to microbubble size. However, non-uniform FITC-antibody attachment was observed (see enlarged images), indicating heterogeneous binding of complement C3/C3b.

It is believed that the PEG overbrush does not completely eliminate macromolecule conjugation to the surface of microbubbles. The heterogeneous fluorescence observed on the shell was another indication of the existence of such microstructural features (FIGS. 18A and 18B).

Control microbubbles without RGD peptide were tested for immunogenicity after human serum incubation (FIG. 19). Three different surface architectures were tested. Significantly lower MFI values were detected for P2K/P5K control than those for P2K control in all microbubble size classes. P5K control microbubbles showed the lowest MFI among all three control samples. For the same methoxy DSPE-PEG surface coverage, the MFI for P5K control 4-5 μm and 6-8 μm microbubbles was only 36% and 31%, respectively, of those for their corresponding P2K control groups. These data indicate complement C3/C3b binding to the underlying phospholipid but that the longer PEG reduces this effect.

It is expected that water-soluble, nonionic PEG can protect colloidal particles, such as microbubbles and liposomes, from aggregation and macromolecule adsorption due to the steric hindrance effect of the polymer brush; each PEG chain forms an impermeable “cloud” over the surface because of its large excluded volume, which inhibits most macromolecules from diffusing into the brush layer. The incorporation of DSPE-PEG5000 into the microbubble shell forces the PEG chains to extend further away from the surface than either the DSPE-PEG2000 alone or the DSPE-PEG2000/5000 mixture, therefore forming a thicker and denser protective layer against complement protein adsorption.

For a given architecture, an increase in complement C3/C3b fixation with microbubble size (FIG. 6) was observed. The increase in MFI was found to be proportional to microbubble surface area, indicating that the surface density of C3/C3b was independent of microbubble diameter. This result was consistent across all microbubble samples, including those with RGD targeting ligands (see below). In addition to the higher total complement fixation, the larger microbubbles could also render them more susceptible to splenic clearance, possibly resulting in shorter circulation persistence. The echoes from untargeted 6-8 μm microbubbles persist much longer than their 1-2 μm or 4-5 μm counterparts, suggesting other mechanisms, such as gas core dissolution, may be at play.

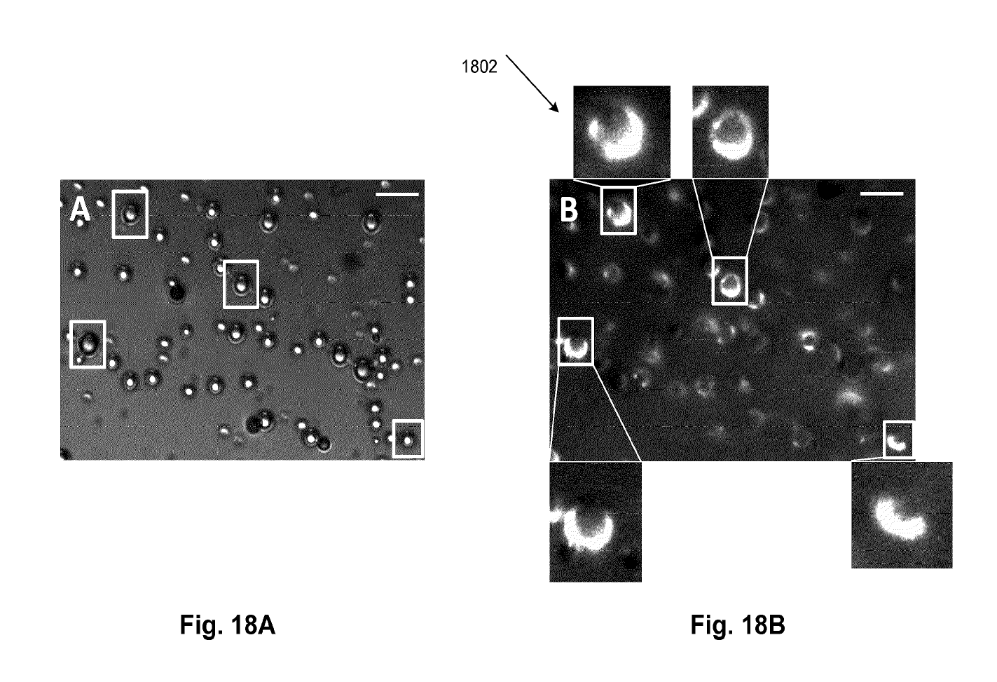

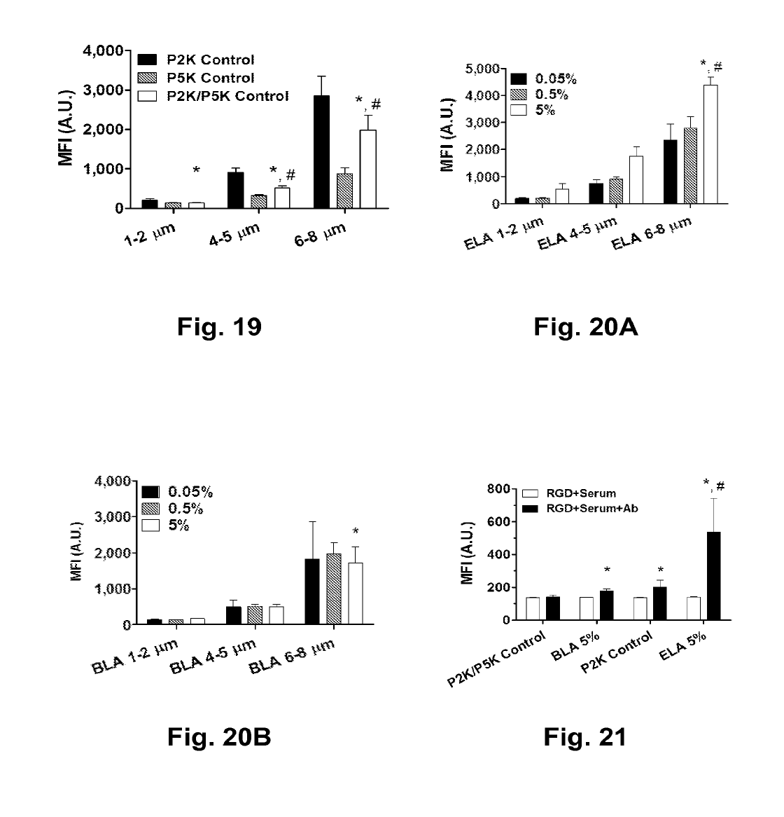

Complement fixation on targeted microbubbles was tested. FIGS. 20A and 20B show the dependence of complement C3/C3b binding on RGD peptide surface density. Targeted ELA microbubbles showed more C3/C3b binding than targeted BLA microbubbles for all RGD peptide surface coverages. For targeted ELA microbubbles, as the amount of conjugated RGD peptide increased, the binding of C3/C3b increased accordingly, indicating a correlated immune response. Complement binding was linearly dependent on microbubble surface area, and the slope (change of MFI per μm2) increased as the ROD surface density increased However, this trend was not seen for targeted BLA microbubbles. For each size class, the MFI did not increase significantly even when the amount of conjugated RGD peptide was increased by two orders of magnitude. These results agreed with findings that the buried-ligand architecture protects the targeting ligands by inhibiting the adsorption of macromolecules to the microbubble surface. Since the extent of opsonization dictates the degree of complement activation, it is concluded that BLA microbubbles triggered less immune recognition than their ELA counterparts.

To further investigate the role of targeting ligand presentation on human complement C3/C3b binding, MFI values were compared for targeted and control microbubbles with the same surface PEG brush layer configurations (FIG. 21). The 1-2 μm size range was used to represent the entire population of microbubbles. The exposed-ligand architecture led to a significant increase in complement activity compared to the P2K control (38-fold increase for ELA 5% vs 15-fold increase for P2K control). This increase in C3/C3b binding may be due to the presence of RGD peptide on the surface, which interacts with complement proteins in serum C3 molecules contain unstable thioester bonds upon cleavage of C3a from C3b RGD peptides contain such nucleophilic groups (eg, the carbonyl group on Asp and the amino group on Arg), which could trigger the immobilization of C3/C3b molecules on the ELA microbubble surface and activate the alternative pathway. The addition of RGD peptide to the surface of BLA microbubbles similarly led to a significant increase in C3/C3b binding. However, when compared to ELA microbubbles, the increase was much lower (13-fold increase for BLA 5% vs no increase for P5K control). Such a small difference in MFI suggested that the buried-ligand architecture indeed partially inhibited the binding of C3/C3b to the microbubble surface and decreased the immunogenicity of targeted microbubbles.

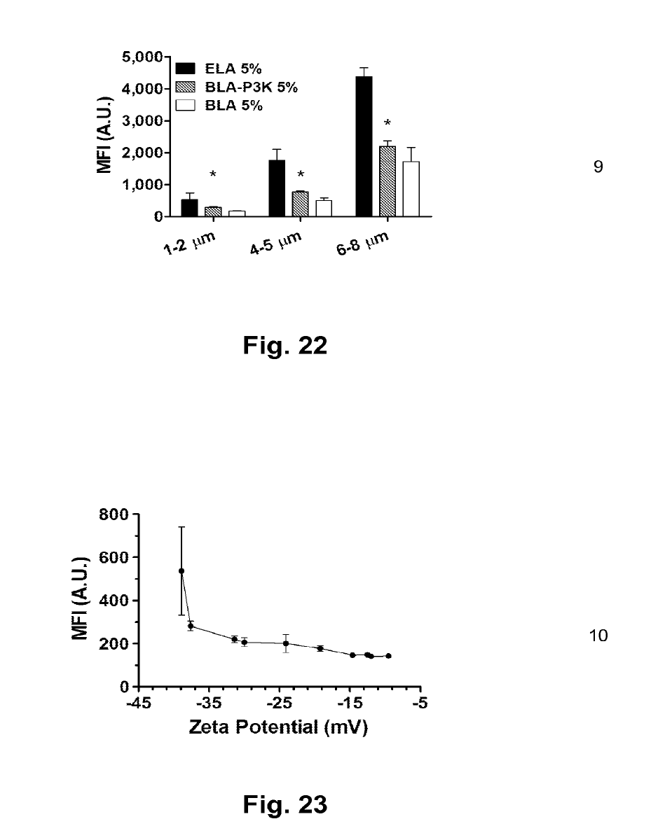

To further illustrate the protective role of PEG chains, the MFI for 5% conjugated RGD peptides for microbubbles were compared with different overbrush lengths (FIG. 22). In addition to the ELA and BLA-P5K design, a different bimodal brush layer using DSPE-PEG3000 to form a shorter PEG overbrush was tested for complement C3/C3b binding. For all three size ranges, BLA-P3K microbubbles showed a measured MFI value that fell between the values detected for ELA and BLA-P5K designs when the same amount of RGD peptide was conjugated to the surface For 6-8 μm microbubbles, there was no significant difference in C3/C3b binding between targeted BLA-P3K and BLA-P5K microbubbles.

The buried-ligand architecture did not completely inhibit the binding of complement C3/C3b to the targeted microbubble surface, presumably due to the phase separation of the phospholipid species in the lipid monolayer coating the microbubble shell. When compared to targeted ELA microbubbles with the same amount of RGD conjugated to the surface, the amount of C3/C3b binding for BLA microbubbles was significantly reduced (˜−52% and ˜−68% for BLA-P3K and BLA-P5K, respectively, for 5% RGD peptide). In embodiments, the combination of the PEG overbrush shielding with the RGD peptide and inhibiting C3/C3b fixation on the microbubble surface may result in reduced complement activation. The buried-ligand architecture successfully protects RGD peptides on the surface of microbubbles from complement recognition, and targeted BLA microbubbles are significantly less immunogenic than ELA microbubbles in vitro.

The effect of surface charge on human complement fixation FIG. 23 shows the human complement C3/C3b binding plotted against the measured average zeta potential (Table 2) for all microbubble formulations within the 1-2 μm diameter range. A weak correlation between microbubble zeta potential and complement C3/C3b fixation was observed: as the negative zeta potential increased, there was more C3/C3b binding on the microbubble surface.

TABLE 1
Microbubble compositions.

| | Phospholipid Composition (mol %) | | | | |
|---|---|---|---|---|---|
| | | | DSPE- | DSPE- | |
| | | DSPE- | PEG2000- | PEG2000- | DSPE- |
| Experiment | DSPC | PEG2000 | A | B | PEG5000 |
| ELA Control | 90 | 10 | — | — | — |
| (no ligand) | | | | | |
| ELA-biotin | 90 | 8 | — | 2 | — |
| ELA-amine | 90 | 8 | 2 | — | — |
| BLA control | 90 | — | — | — | 10 |
| (no ligand) | | | | | |
| BLA-biotin | 90 | — | — | 2 | 8 |
| BLA-amine | 90 | — | 2 | — | 8 |

TABLE 2
Summary of best-fit values obtained for NHS-FITC
ligand binding kinetics curves using a pseudo-
first order kinetics reaction (Equation 1).

| NHS-FITC Binding | |
|---|---|
| ELA | BLA |

| Sample Size | Yₘₐₓ | k_{obs} | Yₘₐₓ | k_{obs} |
|---|---|---|---|---|
| Range | (AFU*) | (hr⁻¹) | (AFU*) | (hr⁻¹) |
| 1-2 μm | 1.1 × 10⁴ | 0.67 | 9.6 × 10³ | 0.65 |
| 4-5 μm | 7.5 × 10⁴ | 0.66 | 6.0 × 10⁴ | 0.64 |
| 7-8 μm | 1.9 × 10⁵ | 0.64 | 1.7 × 10⁵ | 0.58 |

*Arbitrary fluorescence unit

TABLE 3
Summary of best-fit values obtained for SA-FITC
ligand binding kinetics curves using a pseudo-
first order kinetics reaction (Equation 1).

| SA-FITC Binding | |
|---|---|
| ELA | BLA |

| Sample Size | Yₘₐₓ | k_{obs} | Yₘₐₓ | k_{obs} |
|---|---|---|---|---|
| Range | (AFU*) | (hr⁻¹) | (AFU*) | (hr⁻¹)) |
| 1-2 μm | 6.3 × 10³ | ~ | 4.8 × 10³ | 1.80 |
| 4-5 μm | 7.7 × 10⁴ | ~ | 4.0 × 10⁴ | 2.18 |
| 7-8 μm | 2.5 × 10⁵ | ~ | 1.1 × 10⁵ | 1.98 |

*Arbitrary fluorescence unit

TABLE 4
Summary of best-fit values obtained for normalized
FITC ligand binding kinetics curves.
All MFI values were normalized by the corresponding
best-fit Yₘₐₓ values listed in Table 2 and 3.

| | NHS-FITC Binding | | SA-FITC Binding | |
|---|---|---|---|---|
| Sample Size | ELA k_{obs} | BLA k_{obs} | ELA k_{obs} | BLA k_{obs} |
| Range | (hr⁻¹) | (hr⁻¹) | (hr⁻¹) | (hr⁻¹) |
| 1-2 μm | 0.63 | 0.64 | ~ | 1.40 |
| 4-5 μm | 0.62 | 0.63 | ~ | 1.58 |
| 7-8 μm | 0.61 | 0.59 | ~ | 1.48 |

1-70. (canceled)

71. A method of post-production labelling of microbubbles, comprising:
forming size-isolated microbubbles, each microbubble having a lipid shell with polymer spacers attached thereto, each polymer spacer being tethered at one end to the lipid shell and having a first attachment component at the other, each polymer spacer being interspersed and surrounded by PEG brushes having respective lengths greater than a length of the polymer spacer,
the forming including selecting microbubbles having diameters within a range of 1-2 μm from a polydisperse distribution having a multimodal profile using differential centrifugation, the selected microbubbles falling within a size distribution having a size distribution profile (number percentage versus microbubble diameter) that is distinct from the multimodal profile of the polydisperse distribution, the size distribution profile being characterized by a single peak, as measured by an optical particle counter, that is within the range of 1-2 μm;
generating from the selected microbubbles a microbubble cake;
storing the microbubble cake;
recovering the size-isolated microbubbles in solution by diluting the microbubble cake; and
attaching by post-labelling a second attachment component to the recovered size-isolated microbubbles,
wherein the attaching includes loading of the second attachment after the microbubble production, and diffusing the second attachment component through a steric overbrush formed by the shielding component.

72. The method of claim 71, wherein the forming includes incorporating a shielding component in the microbubbles and the shielding and first attachment components include PEG chains.

73. The method of claim 71, wherein the size-isolated microbubbles include a surface of amphiphilic phospholipids that are self-assembled in the forming to form a lipid monolayer shell.

74. The method of claim 71, further comprising using the size-isolated microbubbles as a contrast agent by attaching them to a material with an affinity to the second attachment component and inspecting the material using ultrasound.

75. The method of claim 71, further comprising injecting the microbubbles into a living animal and focusing ultrasound within the living animal.

76. The method of claim 75, wherein the ultrasound generates a tissue-destroying effect for treatment of the living animal.

77. The method of claim 75, further comprising using ultrasound to ameliorate attachment of the microbubbles to native tissue of the living animal.

78. The method of claim 75, further comprising injecting the microbubbles into a living animal and focusing ultrasound within the living animal.

79. The method of claim 75, wherein the second attachment component selectively binds to an angiogenic material.

80. A method of post-production labelling of microbubbles, comprising:
providing a microbubble cake;
the microbubble cake comprising:
microbubbles having lipid shells each with polymer spacers attached to the lipid shell formed in a cake;
each polymer spacer being tethered at one end to the lipid shell and having a primary binding material at the other end;
the polymer spacers of each microbubble being interspersed and surrounded by PEG brush with a length greater than a length of the polymer spacer;
the microbubbles falling within a size distribution resulting from differential centrifugation, a majority of the microbubbles having diameters within a range of 1-2 μm, the size distribution having a size distribution profile (number percentage versus microbubble diameter) that is distinct from a multimodal profile of a polydisperse distribution generated by ultrasonic agitation or shaking without differential centrifugation, said size distribution profile being characterized by a single peak, as measured by an optical particle counter, that is within the range of 1-2 μm; and
the primary binding material attaches to a secondary binding material upon dilution of the microbubble cake to produce microbubbles in solution which are infusible;
diluting the microbubble cake; and
attaching the secondary binding material to said primary binding material to produce an infusible microbubble solution,
wherein the attaching includes loading of the second binding material after the size selected microbubble production, and diffusing the second binding material through a steric overbrush formed by the shielding component.

81. The method of claim 80, wherein the secondary binding material is selected for its affinity to a target particle in the blood of an animal.

82. The method of claim 81, wherein the target particle has a size of about 1 kDa or less.

83. The method of claim 81, wherein the target particle has a size of less than 10 kDa.

84. The method of claim 81, wherein the secondary binding material selectively binds to an angiogenic material.

85. The method of claim 80, wherein prior to the diluting and the attaching, the microbubble cake is stored in a sealed container, which is different from a container in which the microbubble cake was first generated, to maintain the cake in a sterile condition.

86. The method of claim 80, wherein the providing the microbubble cake comprises:
generating a population of microbubbles having the polydisperse distribution with the multimodal profile;
isolating microbubbles having a size in a range of 1-2 μm from the population; and
forming the isolated microbubbles into the microbubble cake.

87. A method of post-production labelling of microbubbles, comprising:
injecting microbubbles having ligands on the surface thereof and buried below a steric barrier tethered to the microbubbles, the ligands having an affinity for both target and non-target materials in the fluid;
preventing attachment of the non-target material while binding target material to the ligands by diffusing the target material through the steric barrier;
eliminating the bound target material,
wherein the steric barrier is size-selective to permit the passage of small target materials and block the passage of non-target materials based on size.

88. The method of claim 87, wherein the fluid includes a biological liquid.

89. The method of claim 87, wherein the target material is a drug.

90. The method of claim 87, wherein the attachment components and ligands are atoms, molecules, or portions thereof that generate an attractive molecular force with specific atoms, molecules, or portions thereof.
